(19) United States
(12) Patent Application Publication
(10) Pub. No.: US 2016/0298162 A1
(43) Pub. Date: Oct. 13, 2016

(54) METHOD FOR DETECTING VIABLE CELLS IN A CELL SAMPLE

(71) Applicant: CHARLES RIVER LABORATORIES, INC., Wilmington, MA (US)

(72) Inventors: Eric STIMPSON, Charleston, SC (US); Norman R. WAINWRIGHT, Johns Island, SC (US)

(21) Appl. No.: 15/034,278

(22) PCT Filed: Nov. 4, 2014
(86) PCT No.: PCT/US2014/063957

§ 371 Date: May 4, 2016

Related U.S. Application Data
(60) Provisional application No. 61/899,580, filed on Nov. 4, 2013.

(51) Int. Cl. C12Q 1/06; G01N 33/52; C12Q 1/10

(52) U.S. Cl. CPC C12Q 1/06; C12Q 1/10; G01N 33/52

(57) ABSTRACT

The invention relates to a method for determining the presence and/or amount of viable cells in a cell containing sample, for example, a fluid sample, using a dye precursor selected from the group consisting of a bioactivatable tetrazolium dye and a bioactivatable esterase dye, where the dye precursor is converted into a fluorescent label by a viable cell.

### CROSS-REFERENCE TO RELATED APPLICATIONS

This application claims the benefit of and priority to U.S. Provisional Patent Application No. 61/899,580, filed Nov. 4, 2013, the entire disclosure of which is incorporated by reference herein in its entirety.

### FIELD OF THE INVENTION

This invention relates generally to method for determining the presence and/or amount of viable cells in a sample.

### BACKGROUND

Microbial contamination by, for example, Gram positive bacteria, Gram negative bacteria, and fungi, for example, yeasts and molds, may cause severe illness and, in some cases, even death in human and animal subjects. Manufacturers in certain industries, for example, food, water, cosmetic, pharmaceutical, and medical device industries, must meet exacting standards to verify that their products do not contain levels of microbial contaminants that would otherwise compromise the health of a consumer or recipient. These industries require frequent, accurate, and sensitive testing for the presence of microbial contaminants to meet certain standards, for example, standards imposed by the United States Food and Drug Administration or Environmental Protection Agency.

Depending upon the situation, the ability to distinguish between viable and non-viable cells can also be important. For example, during the manufacture of pharmaceuticals and biologics, it is important that the water used in the manufacturing process is sterile and free of contaminants. Furthermore, it is important that water contained in medicines (for example, liquid pharmaceutical and biological dosage forms, for example, injectable dosage forms) and liquids (for example, saline) that are administered to a subject, for example, via non-parenteral routes, is also sterile and free of contaminants. On the other hand, the presence of some viable microorganisms in drinking water may be acceptable up to a point. In order to be potable, drinking water must meet exacting standards. Even though microorganisms may be present in the water supply the water may still be acceptable for human consumption. However, once the cell count exceeds a threshold level, the water may no longer be considered safe for human consumption. Furthermore, the presence of certain predetermined levels of microorganisms in certain food products (for example, fresh produce) and drinks (for example, milk) may be acceptable. However, once those levels have been exceeded the food or drink may be considered to have spoiled and no longer be safe of human consumption.

Traditional cell culture methods for assessing the presence of microbial contamination and/or the extent of microbial contamination can take several days to perform, which can depend upon the organisms that are being tested for. During this period, the products in question (for example, the food, drink, or medical products) may be quarantined until the results are available and the product can be released. As a result, there is a need for systems and methods for rapidly detecting (for example, within hours or less) the presence and/or amount of microbial contaminants, in particular, viable microbial contaminants, in a sample.

### SUMMARY

The invention provides a method for detecting the presence and/or quantity of viable cells (for example, prokaryotic cells or eukaryotic cells) in a liquid sample. The method can be used to measure the bioburden (for example, to measure the number and/or percentage and/or fraction of viable cells (for example, viable microorganisms, for example, bacteria, yeast, and fungi)) of a particular sample of interest. In addition, the staining and detection procedures described herein, can be used to detect individual viable cells, for example, individual microorganisms, when captured on a membrane

The present invention is based, in part, upon the discovery of specific fluorescent dyes (which are in the form of substantially non-fluorescent precursors) that are effective viability stains in the detection methods described herein. For example, once the cells are captured on a permeable membrane, the cells can be stained using a dye precursor selected from the group consisting of a bioactivatable tetrazolium dye and an esterase-activatable dye. The dyes used herein are particularly effective for the detection of individual viable cells because of their brightness once photoexcited by light of the appropriate wavelength relative to background fluorescence of the membrane. The dye precursor, prior to conversion in viable cells, is substantially non-fluorescent. As a result, the use of the dyes permits the detection of individual viable cells relative to non-viable cells.

In one aspect, the invention provides a method of detecting the presence and/or quantity of viable cells in a liquid sample to be tested. The method comprises: (a) exposing cells retained by at least a portion of a substantially planar porous membrane after passing the liquid sample through the portion of the substantially planar porous membrane to a dye precursor selected from the group consisting of a bioactivatable tetrazolium dye and an esterase-activatable dye, under conditions so that the dye precursor is converted to a fluorescent label by a viable cell; (b) scanning the portion of the porous membrane by rotating the porous membrane relative to a detection system comprising (i) a light source emitting a beam of light of a wavelength adapted to excite the fluorescent label to produce an emission event, and (ii) at least one detector capable of detecting the emission event, thereby to interrogate a plurality of regions of the planar porous membrane and to detect emission events produced by excitation of fluorescent label associated with any viable cells; and (c) determining the presence and/or quantity of viable cells captured by the membrane based upon the emission events detected in step (b).

The scanning step can comprise tracing at least one of a nested circular pattern and a spiral pattern on the porous membrane with the beam of light. It is understood that during the scanning step, the porous membrane may move (for example, via linear translation and/or rotation about a rotation axis) while the detection system remains static. Alternatively, the detection system may move (for example, via linear translation) while the porous membrane rotates about a single point (for example, the porous membrane rotates about a rotation axis at a single location). Alternatively, it is possible that the both the porous membrane and the detection may move and that their relative positions can be measured with respect to one another.

The detection method can be performed on single cells, clusters of cells or colonies (for example, microcolonies) of cells. In one embodiment, the detection method can be used to detect an individual viable cell disposed upon a membrane. In another embodiment, the detection method can be used to detect microcolonies of viable cells disposed upon a membrane. This can be useful when detecting cells that grow slowly and/or have a lower metabolic activity than fast growing cells such as E. coli.

Under certain circumstances, for example, in order to increase the sensitivity of the assay, it may be desirable to culture the cells under conditions that permit cell proliferation prior to and/or during and/or after exposing the cells to the fluorescent dye precursor. The culture conditions, including, the choice of the growth media, the temperature, the duration of the culture, can be selected to permit at least one of cells in the sample to have one or more cell divisions.

Furthermore, it is understood that a porous membrane having cells disposed thereon can be placed upon growth media containing the dye precursor and/or having the dye precursor disposed upon a surface adjacent the porous membrane and incubated under conditions and for a time sufficient to permit the dye precursor to permeate the membrane and enter viable cells disposed upon the membrane. Thereafter, the dye precursor is converted into a fluorescent dye by the viable cells. This approach maintains the integrity of the colonies, which may otherwise be disturbed or destroyed if the dye precursor is applied directly to the colonies.

In certain embodiments, the beam of light used to excite the fluorescent dye created from the precursor has a wavelength in the range of from about to 350 nm to about 1000 nm, from about 350 nm to about 900 nm, from about 350 nm to about 800 nm, from about 350 nm to about 700 nm, or from about 350 nm to about 600 nm. For example, the wavelength of excitation light is at least in one range from about 350 nm to about 500 nm, from about 350 nm to about 500 nm, from about 350 nm to about 600 nm, from about 400 nm to about 550 nm, from about 400 nm to about 600 nm, from about 400 nm to about 650 nm, from about 450 nm to about 600 nm, from about 450 nm to about 650 nm, from about 450 nm to about 700 nm, from about 500 nm to about 650 nm, from about 500 nm to about 700 nm, from about 500 nm to about 750 nm, from about 550 nm to about 700 nm, from about 550 nm to about 750 nm, from about 550 nm to about 800 nm, from about 600 nm to about 750 nm, from about 600 nm to about 800 nm, from about 600 nm to about 850 nm, from about 650 nm to about 800 nm, from about 650 nm to about 850 nm, from about 650 nm to about 900 nm, from about 700 nm to about 850 nm, from about 700 nm to about 900 nm, from about 700 nm to about 950 nm, from about 750 to about 900 nm, from about 750 to about 950 nm or from about 750 to about 1000 nm. Certain ranges include from about 350 nm to about 600 nm and from out 600 nm to about 750 nm.

In certain embodiments, the fluorescent dye can be excited to undergo fluorescence by radiation from a red laser, for example, a red laser that emits light having a wavelength in the range of 620 nm to 640 nm.

Depending upon the fluorescent dye employed, the optical detector can detect emitted light in a range of from about 350 nm to about 1000 nm, from about 350 nm to about 900 nm, from about 350 nm to about 800 nm, from about 350 nm to about 700 nm, or from about 350 nm to about 600 nm. For example, the fluorescent emission can be detected within a range from about 350 nm to 550 nm, from about 450 nm to about 650 nm, from about 550 nm to about 750 nm, from about 650 nm to about 850 nm, or from about 750 nm to about 950 nm, from about 350 nm to about 450 nm, from about 450 nm to about 550 nm, from about 550 nm to about 650 nm, from about 650 nm to about 750 nm, from about 750 nm to about 850 nm, from about 850 nm to about 950 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to 700 nm, from about 700 nm to about 750 nm, from about 750 nm to about 800 nm, from about 800 nm to about 850 nm, from about 850 nm to about 900 nm, from about 900 nm to about 950 nm, or from about 950 nm to about 1000 nm. In certain embodiments, the emitted light is detected in the range from about 660 nm to about 690 nm, from about 690 nm to about 720 nm, and/or from about 720 nm to about 850 nm. In certain other embodiments, the emitted light is detected in the range from about 490 nm to about 540 nm and/or from about 590 nm to about 700 nm.

The membrane can be of any of a variety of shapes, for example, circular, annular, ovoid, square, rectangular, elliptical, etc., and can have some portion or all of one side exposed for cell retention. Moreover, the membrane may form one or more apertures therein to accommodate a mask, and may be formed from several separate membranes assembled together with the mask or other structural element. In one embodiment, the membrane may be in the shape of a disc, for example, a substantially planar disc. Depending upon the detection system employed, the porous membrane is substantially non-autofluorescent when exposed to light having a wavelength in the range from about 350 nm to about 1000 nm. Furthermore, depending upon the detection system used, the region of the porous membrane that contains the cells to be detected is substantially planar having a flatness tolerance of up to about 100 μm (i.e., within ±50 μm). Furthermore, the porous membrane can define a plurality of pores having an average diameter less than about 1 μm so as to permit fluid to traverse the porous membrane while retaining cells thereon. The porous membrane can have a thickness in a range selected from the group consisting of from 1 μm to 3,000 μm, from 10 μm to 2,000 μm, and from 100 μm to 1,000 μm.

In certain embodiments, the cell capture system further comprises a fluid permeable support member (optionally a substantially planar fluid permeable support member) adjacent at least a portion of a second opposing surface of the membrane. The fluid permeable support, for example, in the form of a porous plastic frit, retains enough fluid to maintain moisture in the porous membrane disposed adjacent the permeable support, which in certain embodiments, can be important to maintain the viability of cells retained by the porous membrane. The support member can have a thickness in a range selected from the group consisting of from 0.1 mm to 10 mm, from 0.5 mm to 5 mm, and from 1 mm to 3 mm. In addition, the permeable support member may maintain the flatness of the membrane disposed thereon to a flatness tolerance of up to about 100 μm.

In addition, it is possible to include a positive control for the detection system. As a result, the method can further comprise combining the cells in the sample with a plurality of fluorescent particles that emit a fluorescent signal upon activation by light having a wavelength in the range of from about 350 nm to about 1000 nm. Thereafter, a fluorescent signal produced by one or more of the fluorescent particles can be detected at the same time any viable cells are being detected by the detection system.

The method can be used to determine the quantity of viable cells in at least a portion of the liquid sample. Furthermore, the detection system can be used to determine the location(s) of the viable cells on the permeable membrane. In order to measure the determine the locations of the cells, the cell capture system optionally further comprises a register (for example, line, spot, or other mark, indicia or structural feature) associated with the membrane so as to permit the determination of the location of cells retained on at least a portion of the planar membrane. For a disc shaped membrane, polar coordinates (i.e., radial “r” and angular “0” coordinate locations) may be suitable.

After the detection step, the viable cells can be cultured under conditions that permit growth and/or proliferation of the viable cells captured by the porous membrane. The genus and/or species of the viable organisms can be determined by standard procedures, for example, microbiological staining and visualization procedures, or molecular biology procedures, for example, amplification procedures including polymerase chain reaction, ligase chain reaction, rolling circle replication, and the like, and by nucleic acid sequencing.

These and other objects, along with advantages and features of the embodiments of the present invention herein disclosed, will become more apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A is a schematic representation of an exemplary detection system that can be used to determine the presence and/or amount of viable cells in a cell sample;

FIG. 1B is a schematic perspective view of an exemplary detection system with a door in a closed position;

FIG. 1C is a schematic perspective view of the exemplary detection system of FIG. 1B with the door in an open position;

FIG. 1D is a schematic perspective view of the exemplary detection system of FIG. 1B with a touchscreen in a raised position;

FIG. 2A is a schematic top view of an exemplary membrane assembly;

FIG. 2B is a schematic, exploded side view of the membrane assembly of FIG. 2A.

FIGS. 3A and 3B are schematic representations of exemplary membrane assemblies;

FIG. 4A is a schematic, exploded perspective view of an exemplary membrane assembly having a permeable membrane and a fluid permeable support member;

FIG. 4B is a schematic side view of the exemplary permeable membrane assembly of FIG. 4A;

FIG. 5A is a schematic perspective view of an exemplary cell capture cup and a corresponding base;

FIG. 5B is a schematic partial cut-away view of the cup and base of FIG. 5A showing a membrane assembly;

FIG. 5C is a schematic perspective view of the cup, base, and membrane assembly of FIG. 5B in an unassembled state;

FIG. 5D is a schematic perspective view of the base of FIG. 5A;

FIG. 5E is a schematic partial cross-sectional view of the cup and base of FIG. 5B with a different membrane holder assembly and posts from a separate holder;

FIGS. 6A-6D are schematic perspective, side, top, and bottom views, respectively, of a cup assembly having a lid, a cup, a membrane, and a base;

FIG. 6E is a schematic bottom perspective view of the lid of FIG. 6A;

FIG. 6F is a schematic side view of the lid of FIG. 6A;

FIGS. 7A-7D are schematic perspective, side, top and bottom views, respectively, of a cup member shown in the cup assembly of FIG. 6A;

FIGS. 8A-8D are schematic perspective, top, bottom, and side views, respectively, of the base of the cup assembly of FIG. 6A;

FIG. 9A is a schematic perspective view of the base of FIG. 8A showing a partial cut away view of the membrane and underlying permeable support member;

FIGS. 9B-9D are schematic top, bottom, and side views of the base (complete membrane and underlying permeable support member) of FIG. 9A;

FIG. 10A is a schematic exploded perspective view of the cup assembly of FIG. 6A;

FIG. 10B is a schematic cross-sectional view of the cup assembly of FIG. 6A, without a membrane and a permeable support member;

FIG. 10C is a schematic cross-sectional view of the base of FIG. 9A with a membrane, a permeable support member, and a base lid;

FIG. 11A depicts a process for capturing cells on a permeable membrane;

FIG. 11B depicts a schematic exploded perspective view of a chuck, stage, base, support member, membrane, and base lid components;

FIG. 12 is a schematic representation of the emission spectra of a first fluorescent dye and a second fluorescent dye illustrating the embodiment where the first fluorescent dye has a high-intensity emission at a wavelength substantially different from the high-intensity emission by the second fluorescent dye (e.g., the λₘₐₓ₁ of the emission spectrum of the first fluorescent dye is distinct from the λₘₐₓ₂ of the emission spectrum of the second fluorescent dye) so that the emission signals from both fluorescent dyes can be measured substantially independently;

FIG. 13A is a schematic exploded cross-sectional side view of an exemplary membrane holder for use with the system of FIG. 1A;

FIG. 13B is a schematic of a configuration of magnets for use with the membrane holder (stage) of FIG. 13A;

FIG. 13C is a schematic exploded perspective view of the membrane holder (stage) of FIG. 13A with a membrane assembly and chuck;

FIG. 13D is a schematic perspective view of the membrane holder, membrane assembly, and chuck of FIG. 13C in an assembled configuration;

FIGS. 14A-14C are schematic perspective, top, and bottom views, respectively, of an exemplary stage;

FIGS. 15A-15D are schematic perspective, top, bottom, and side views, respectively, of an exemplary chuck;

FIG. 16A is a schematic perspective view of an exemplary membrane holder (stage) for receiving a base;

FIG. 16B is a schematic perspective view of an exemplary base for use with the membrane holder of FIG. 16A;

FIG. 16C is a schematic perspective view of the exemplary membrane holder of FIG. 16A and the base of FIG. 16B in an unassembled configuration;

FIG. 16D is a schematic perspective view of the exemplary membrane holder of FIG. 16A and the base of FIG. 16B in an assembled configuration showing posts extending from the membrane holder and passing through operatives defined by the base;

FIG. 17 is a schematic representation of viable (live) and non-viable (dead) cells following staining with a non-fluorescent dye precursor that becomes fluorescent within a viable cell but does not become fluorescent in a non-viable cell;

FIG. 18 is a schematic representation of a region of a permeable membrane showing viable and non-viable cells stained with an exemplary viability staining system shown in FIG. 17;

FIG. 19 is a fluorescent image of viable cells (E. coli) captured on a permeable membrane, stained with an exemplary esterase substrate and detected as fluorescent events on a rotating disk using the detection system shown in FIG. 1A;

FIG. 20 is a fluorescent image of viable cells (Micrococcus luteus) captured on a permeable membrane, stained with an exemplary esterase substrate and detected as fluorescent events on a rotating disk using the detection system shown in FIG. 1A;

FIG. 21 is a fluorescent image of viable cells (Staphylococcus aureus) captured on a permeable membrane, stained with an exemplary bioactivatable tetrazolium dye and detected as fluorescent events on a rotating disk using the detection system shown in FIG. 1A; and

FIGS. 22A and 22B are fluorescent images of E. coli microcolonies (FIG. 22A) or as an individual E. coli cell (FIG. 22B) on a rotating membrane after staining with an exemplary bioactivatable tetrazolium dye and detected using the detection system shown in FIG. 1A.

### DESCRIPTION

The detection method can be used to determine the presence and/or amount of viable cells in a cell containing sample (e.g., a liquid sample) and, in particular, can be used to determine the bioburden (e.g., to measure the number and/or percentage and/or fraction of viable cells in a sample) of a particular sample of interest. The method can be used to measure the bioburden of cells in a liquid sample (e.g., a water sample), a comestible fluid (e.g., wine, beer, milk, baby formula or the like), a body fluid (e.g., blood, lymph, urine, cerebrospinal fluid or the like), growth media, a liquid sample produced by harvesting cells from a source of interest (e.g., via a swab) and then dispersing and/or suspending the harvested cells, if any, in a liquid (e.g., buffer or growth media).

It is contemplated that, by using the methods and devices described herein, it will be possible to determine the presence and/or amount of viable cells in sample within less than approximately 6 hours, less than approximately 4 hours, less than approximately 2 hours, less than approximately 1 hour, or even less than approximately 30 minutes after the cells have been captured on a porous membrane of the cell capture system. However, it is contemplated that, depending upon the desired sensitivity, it is possible to culture the cells captured on the porous membrane (e.g., for 15 minutes to several hours) to permit cell proliferation. Nevertheless, by using the devices and methods described herein, even when including a culturing step, it is possible to determine the presence and/or amount of viable cells in a sample faster and/or more reliably than other technologies available in the art.

### (I) Cell Capture System

The cell capture system described herein can be used with an optical detection system that detects the presence of viable cells. The results can be used to measure the bioburden (e.g., to measure the number and/or percentage and/or fraction of viable cells in a sample) of a particular sample of interest. Exemplary detection systems are described, for example, in International Patent Application No. PCT/IB2010/054965, filed Nov. 3, 2010, U.S. patent application Ser. No. 13/034,402, filed Feb. 24, 2011, International Patent Application No. PCT/IB2010/054966, filed Nov. 3, 2010, U.S. patent application Ser. No. 13/034,380, filed Feb. 24, 2011, International Patent Application No. PCT/IB2010/054967, filed Nov. 3, 2010, and U.S. patent application Ser. No. 13/034,515, filed Feb. 24, 2011.

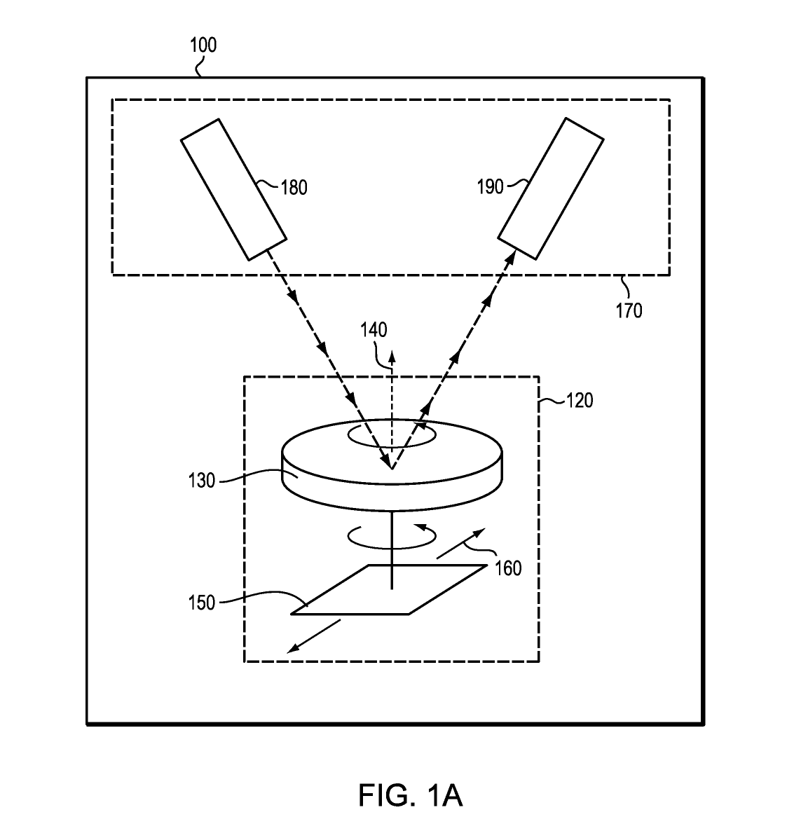

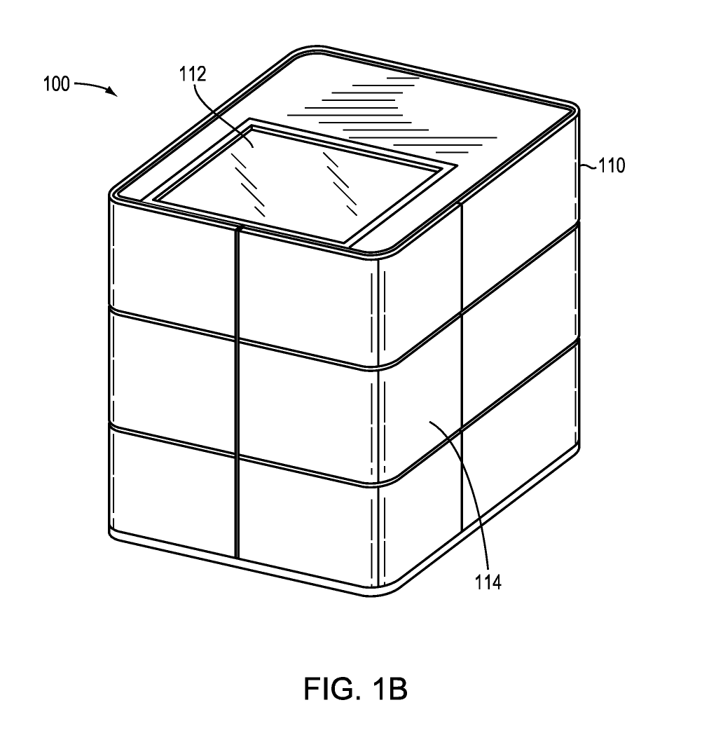

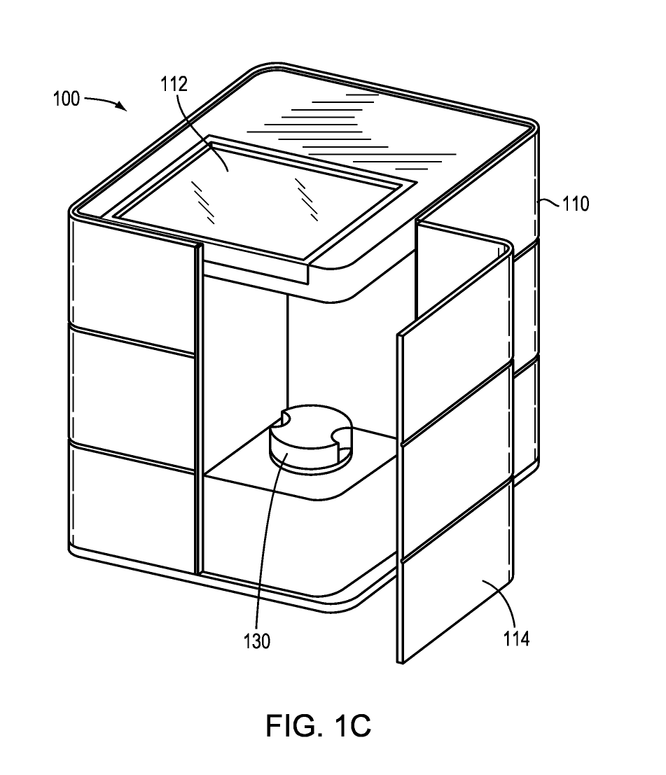

One embodiment of an exemplary system 100, as shown schematically in FIG. 1A, comprises a sample assembly 120 comprising (i) a rotating platform 130 upon which a porous membrane having cells disposed thereon rotates about a rotation axis 140, and (ii) a movable platform 150 that translates linearly (see track 160) relative to a detection system 170 that comprises a light source 180 (e.g., a white light source or a laser light source (e.g., a near infrared laser)), and at least one detector 190, for example, a fluorescence detector. A beam of light from light source 180 (excitation light) impinges rotating platform 130 and the planar membrane disposed thereon, while emission light is detected by detector 190. The light source 180 and the detector 190 may be arranged at similar angles relative to the platform 130 as the beam of light will impact and leave the platform 130 at substantially the same angle. In certain circumstances, the detection system consists of a single detector that detects a single wavelength range or that detects multiple wavelength ranges. Alternatively, the detection system consists of multiple detectors, each of which is capable of detecting a different wavelength range.

FIGS. 1B-1D depict the exemplary cell detector system 100 having an enclosure 110 and a display (e.g., a touchscreen) 112. The enclosure 110 is sized to house the rotating platform 130, which may be accessed through a door 114 on the enclosure 110. The enclosure 110 may be manufactured in various shapes and sizes, including in the depicted rectangular prism form that is approximately 10 in.×10 in.×12 in. (l×w×h). Other shapes may be a cube, cylinder, sphere, or other prism, amongst others. While dimensions vary depending on the shape, the enclosure 110 may range in scale from a few inches to several feet, and possibly lesser or greater, depending on the application. FIG. 1B depicts a cell detection system with the door 114 in a closed configuration and FIG. 1C depicts the same system with door 114 in an open configuration to show rotating platform 130. The touchscreen 112 provides a user interface for controlling the operation of the system 100, and may display information regarding the system's 100 current operating parameters. The touchscreen 112 may be adjustable into a more upright position (as depicted in FIG. 1D) in order to facilitate easier operation. In certain embodiments, the touchscreen 112 is only active when in the upright position. In other embodiments, the touchscreen 112 is always active, or only at select times (e.g., when engaged by a user).

It is understood that such detection systems operate optimally when the cells are disposed upon a solid support or otherwise maintained in a substantially planar orientation with a tight flatness tolerance (e.g., within a flatness tolerance of up to about 100 μm (±50 μm), e.g., up to about 10 μm (±5 μm), up to about 20 μm (±10 μm), up to about 30 μm (±15 μm), up to about 40 μm (±20 μm), up to about 50 μm (±25 μm), up to about 60 μm (±30 μm), up to about 70 μm (±35 μm), up to about 80 μm (±40 μm), up to about 90 μm (±45 μm)), so that the cells can be visualized readily by a detection system within a narrow focal plane. A flatness tolerance specifies a tolerance zone defined by two parallel planes within which the surface must lie. For example, where a membrane or a portion of a membrane has a flatness tolerance of up to about 100 μm, each point on the membrane or the portion of the membrane must fall between two parallel planes spaced 100 μm apart. If a dynamic focusing system is employed, it is contemplated that flatness tolerances greater than 100 μm can be tolerated. Accordingly, it can be preferable to use a support system that maintains the membrane and any captured cells in a substantially planar orientation and within a suitably tight flatness tolerance to permit reliable detection. Depending on the detection system and requirements post detection, the support system may be adapted to present and/or maintain planarity of the membrane when dry and/or when wet or moist after cells have been captured on the solid support after passing a cell containing solution through the solid support via pores disposed within the solid support.

A cell capture system useful in the practice of the invention comprise a fluid permeable, planar membrane comprising an exposed first surface, at least a portion of which is adapted to retain cells thereon. The portion can: (i) define a plurality of pores having an average diameter less than about 1 μm so as to permit fluid to traverse the portion of the membrane while retaining cells thereon; and (ii) be substantially non-auto-fluorescent when exposed to light having a wavelength in a range from about 350 nm to about 1000 nm. Furthermore, the portion optionally can have a flatness tolerance of up to about 100 μm. The cell capture system 100 optionally further comprises a register (e.g., line, spot, or other mark, indicia or structural feature) associated with the membrane so as to permit the determination of the location of cells (for example, the viable cells) retained on at least a portion of the planar membrane. For a disc shaped membrane, polar coordinates (i.e., radial “r” and angular “0” coordinate locations) may be suitable.

The membrane can be of any of a variety of shapes, e.g., circular, annular, ovoid, square, rectangular, elliptical, etc., and can have some portion or all of one side exposed for cell retention. Moreover, the membrane may form one or more apertures therein to accommodate a mask and may be formed from several separate membranes assembled together with the mask or other structural element. In one embodiment, the membrane may be in the shape of a disc, e.g., a substantially planar disc. In certain embodiments, the portion of the porous membrane for capturing cells and/or particles is greater than 400 mm², 500 mm², 600 mm², 700 mm², 800 mm², 900 mm² or 1,000 mm². The membrane (e.g., in the form of a disc) can have a thickness in a range selected from the group consisting of approximately from 1 μm to 3,000 μm, from 10 μm to 2,000 μm, and from 100 μm to 1,000 μm.

In certain embodiments, the cell capture system 100 further comprises a fluid permeable support member (for example, a substantially planar fluid permeable support member) adjacent at least a portion of a second opposing surface of the membrane. The fluid permeable support, for example, in the form of a smooth planar porous plastic frit, retains enough fluid to maintain moisture in the porous membrane disposed adjacent the permeable support, which in certain embodiments, can be important to maintain the viability of cells retained on the porous membrane. The support member can have a thickness in a range selected from the group consisting of approximately from 0.1 mm to 10 mm, from 0.5 mm to 5 mm, and from 1 mm to 3 mm.

The porous membrane defines a plurality of pores having an average diameter less than about 1 μm so as to permit fluid to traverse the membrane while retaining cells thereon. In certain embodiments, the average pore diameter is about or less than about 0.9 μm, 0.8 μm, 0.7 μm, 0.6 μm, 0.5 μm, 0.4 μm, 0.3 μm, 0.2 μm, 0.1 μm, or 0.05 μm. In certain embodiments, the average pore diameter is about 0.2 μm, and in other embodiments the average pore diameter is about 0.4 μm. Suitable membranes can be fabricated from nylon, nitrocellulose, polycarbonate, polyacrylic acid, poly(methyl methacrylate) (PMMA), polyester, polysulfone, polytetrafluoroethylene (PTFE), polyethylene and aluminum oxide.

In addition, the porous membrane is substantially non-autofluorescent when exposed to light having a wavelength in the range from about 350 nm to about 1,000 nm. As used herein with reference to the porous membrane, the term “substantially non-autofluorescent when exposed to a beam of light having a wavelength in the range from about 350 nm to about 1,000 nm” is understood to mean that the porous membrane emits less fluorescence than a fluorescently labeled cell or a fluorescent particle disposed thereon when illuminated with a beam of light having a wavelength, fluence and irradiance sufficient to cause a fluorescence emission from the cell or particle. It is understood that a user and/or detector should be able to readily and reliably distinguish a fluorescent event resulting from a fluorescent particle or a fluorescently labeled cell from background fluorescence emanating from the porous membrane. The porous membrane is chosen so that it is possible to detect or visualize a fluorescent particle or a fluorescently labeled cell disposed on such a porous membrane. In certain embodiments, the fluorescence emitted from a region of a porous membrane (e.g., a region having approximately the same surface area as a cell or cell colony or particle being visualized) illuminated with a beam of light may be no greater than approximately 30% (e.g., less than 30%, less than 27.5%, less than 25%, less than 22.5%, less than 20%, less than 17.5%, less than 15%, less than 12.5%, less than 10%, less than 7.5%, less than 5%, or less than 2.5%) of the fluorescence emitted from a fluorescent particle or a fluorescently labeled cell, when measured under the same conditions, for example, using a beam of light with the same wavelength, fluence and/or irradiance.

Suitable membranes that are non-autofluorescent can be fabricated from a membrane, e.g., a nylon, nitrocellulose, polycarbonate, polyacrylic acid, poly(methyl methacrylate) (PMMA), polyester, polysulfone, polytetrafluoroethylene (PTFE), or polyethylene membrane impregnated with carbon black or sputtered with an inert metal such as but not limited to gold, tin or titanium. Membranes that have the appropriate pore size which are substantially non-autofluorescent include, for example, ISOPORE™ membranes (Merck Millipore), NUCLEOPORE™ Track-Etched membranes (Whatman), ipBLACK Track Etched Membranes (distributed by AR Brown, Pittsburgh, Pa.), and Polycarbonate (PCTE) membrane (Sterlitech).

In order to facilitate accurate detection and count estimation of the captured cells, it is beneficial (even essential in some instances, depending on the configuration and capabilities of the detection system) that the membrane is substantially planar (e.g., substantially wrinkle free) during cell detection. As used herein, the term “substantially planar” is understood to mean that an article has a flatness tolerance of less than approximately 100 μm (i.e., within ±50 μm). This is because height imperfections (e.g., wrinkles) may interfere with the optical detection/measurement system, leading to erroneous results. As a result, it can be important for the porous membrane when dry and/or wet and depending on detection conditions), retains a relatively tight flatness tolerance, within the detection capability of the detection system. Various approaches described below allow the porous membrane to be held substantially flat after cells from a sample fluid are captured thereon and other approaches may be apparent to those skilled in the art based on the discussion herein. However, in one embodiment the membrane is maintained within a flatness tolerance of less than approximately 100 μm by placing the membrane upon a fluid permeable support having a membrane contacting surface having a flatness tolerance of less than approximately 100 μm.

In certain embodiments, the cell capture system further comprises a plurality of detectable particles, for example, fluorescent particles. The fluorescent particles can be adapted to be excited by a beam of light having a wavelength at least in a range from about 350 nm to about 1000 nm, a wavelength in a range from about 350 nm to about 600 nm a wavelength in a range from about 600 nm to about 750 nm, or any of the wavelength ranges discussed above. The particles can be used as part of a positive control to ensure that one or more of the cell capture system, the cell capture method, the detection system, and the method of detecting the viable cells are operating correctly.

Depending upon the design of the cell capture system, the particles (for example, fluorescent particles) can be pre-disposed upon at least a portion of the porous membrane or disposed within a well formed in a mask. Alternatively, the particles (for example, fluorescent particles) can be mixed with the liquid sample prior to passing the sample through the porous membrane. In such an approach, the fluorescent particles can be dried in a vessel that the sample of interest is added to. Thereafter, the particles can be resuspended and/or dispersed within the liquid sample. Alternatively, the fluorescent particles can be present in a second solution that is mixed with the liquid sample of interest. Thereafter, the particles can be dispersed within the liquid sample.

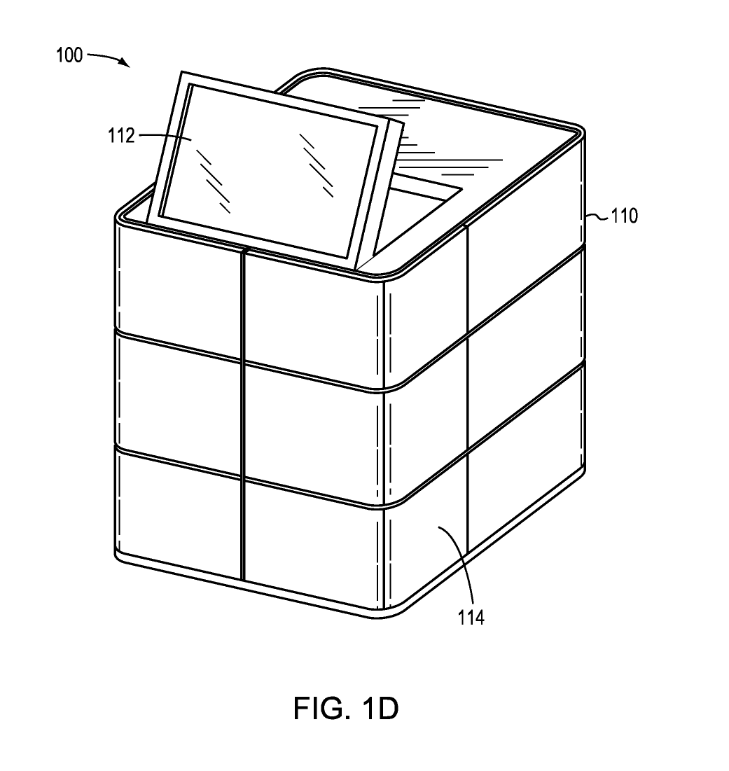

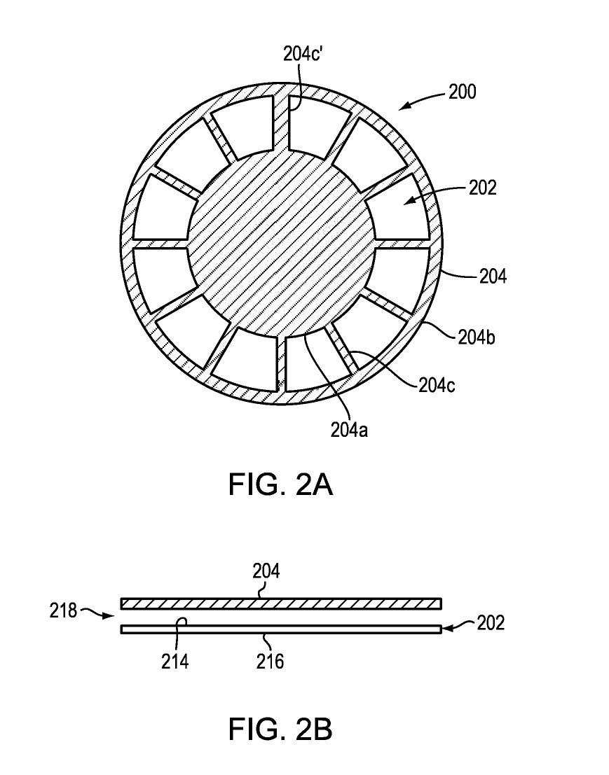

FIG. 2A shows an exemplary membrane assembly 200 comprising a porous planar membrane 202 and a frame (or mask) 204 to hold porous membrane 202 substantially flat, i.e., without allowing the formation of significant wrinkles therein. As shown, frame 204 comprises a central portion 204a connected to a circumferential portion or outer rim 204b via a plurality of spokes (e.g., tensioning spokes) 204c. One of the spokes denoted 204c′ may be thicker than the other spokes 204c and represents a register from which the co-ordinates of cells disposed on the membrane can be measured (for example, r, θ values), where r is the radial distance measured from the axis of rotation and θ is the included angle between (i) a radial line traversing the point of rotation and the cell and (ii) the register 204c′.

Membrane 202 comprises a plurality of pores having an average diameter about or less than about 1 μm, for example, about or less than about 0.9 μm, 0.8 μm, 0.7 μm, 0.6 μm, 0.5 μm, 0.4 μm, 0.3 μm, 0.2 μm, 0.1 μm, or 0.05 μm. As such, when a liquid fluid containing cells and/or particles contacts membrane 202, the fluid can traverse through the membrane via the pores, while the cells and/or particles are retained on a surface of the membrane 202. The membrane 202 is substantially non auto-fluorescent when exposed to light having a wavelength in the range from about 350 nm to about 1000 nm. Moreover, the membrane 202 has a smooth surface having a flatness tolerance no greater than about 100 μm when restrained or configured for detection by the associated detection system.

As shown in FIG. 2B, membrane 202 has a first surface 214 and a second surface 216 that is opposite the first surface. First surface 214 may be affixed to the frame 204, e.g., via an adhesive bonding layer 218. The central portion 204a can be affixed to a central portion of membrane 202. In the embodiment shown, the diameter of the membrane 202 is about the same as that of the outer rim 204b, and as a result, the outer rim 204b is affixed to the perimeter of the membrane 202. The spokes 204c extend radially from the central portion 204a and may be affixed to the membrane 202. This configuration can hold the membrane 202 substantially flat, preventing or minimizing the formation of wrinkles. Furthermore, the formation of wrinkles can also be mitigated or eliminated by applying downward pressure on the central portion 204a, which increases the surface tension in membrane 202.

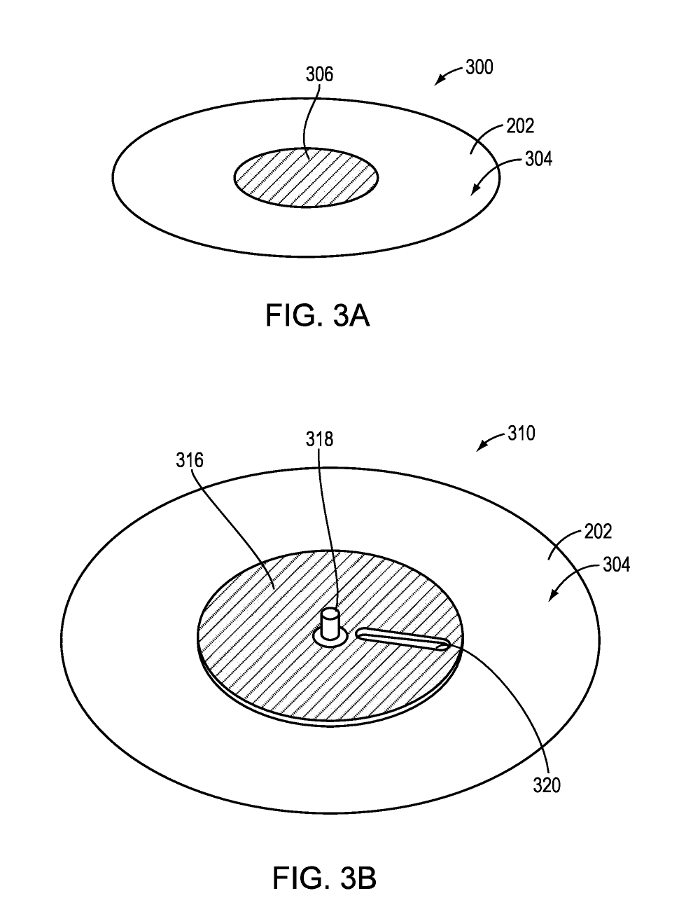

In another approach, as depicted in FIG. 3A, a circular membrane assembly 300 comprises a porous membrane 202 having an upper surface 304. A circular mask 306, affixed to a central portion of the surface 304, holds the membrane 202 substantially flat. In the membrane assembly 300, cells in the fluid sample, if any are present therein, are captured on the exposed portion of the surface 304 that is not covered by the mask 306. Membrane assembly 300 may be disposed on a fluid permeable porous support member, as described below, that may maintain the desired flatness of the membrane during detection. Alternatively or additionally, in order to keep the membrane 202 substantially flat, downward pressure may be applied to the mask 306. Materials suitable for the mask 306 include plastic, polycarbonate, polystyrene, polypropylene, and other materials having water repellant properties.

FIG. 3B depicts a membrane assembly 310 that is similar to the membrane assembly 300 shown in FIG. 3A. A mask 316 is similar to the mask 306, but the mask 316 has a protrusion or nipple 318 that allows a user to pick up the assembly 310 (including the membrane 202) with fingers or forceps, and transfer the assembly 310 to another location, e.g., on a membrane holder. The top surface of the mask 316 also defines a well 320 that may serve as a register so that the location of a particle or cell detected on the surface 304 of the membrane 202 can be described with reference to the location of the well 320. Alternatively or in addition, control particles to be detected may be initially disposed in the well 320. In certain other embodiments, the mask may include either the protrusion or the well, but not both.

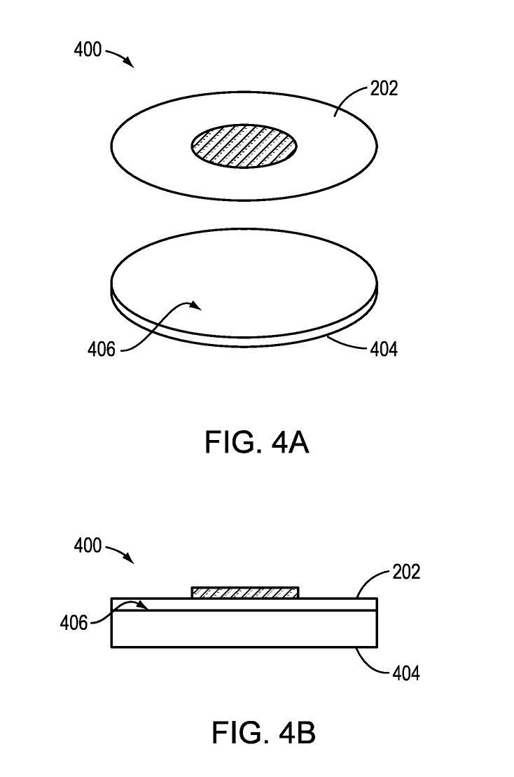

In another approach, as depicted in FIGS. 4A and 4B, the porous membrane 202 may be disposed in a membrane assembly 400 to maintain the porous membrane 202 in a substantially planar configuration without the need for the frame 204 or the masks 306 or 316, by placing the porous membrane 202 upon a fluid permeable, substantially planar solid support member 404. In one embodiment, when the porous membrane 202 is wetted, surface tension between the membrane 202 and the solid support member 404 conforms the bottom surface of the membrane 202 to an upper mating surface 406 of the support member 404. For example, in one embodiment, the support member 404 may be a fluid permeable, solid substantially planar element that keeps membrane 202 in a substantially planar configuration, for example, when the membrane is wetted. The support member 404 is porous, and the upper mating surface 406 is substantially flat and smooth. In another embodiment, the solid support member 404 is coated with a non-toxic adhesive, for example, polyisobutylene, polybutenes, butyl rubber, styrene block copolymers, silicone rubbers, acrylic copolymers, or some combination thereof. When a downward pressure is applied, for example, from a vacuum, the porous membrane 202 becomes loosely adhered to the solid support member 404, which results in the porous membrane conforming to the surface 406 of the solid support member 404. The support member 404 is porous, and the upper mating surface 406 is substantially flat and smooth. For example, in one embodiment, the surface 406 has a flatness tolerance of up to about 100 μm. The diameter of the support member 404 is approximately the same as that of the membrane 202, and preferably the support member 404 has a substantially uniform thickness. The support member can have a thickness in a range selected from the group consisting of approximately from 0.1 mm to 10 mm, from 0.5 mm to 5 mm, and from 1 mm to 3 mm. Materials suitable for making the porous support member 404 include plastic, polycarbonate, high density polyethylene (HDPE), glass, and metal. In one embodiment, the support member 404 is fabricated by sintering plastic particles made from poly (methyl methacrylate) having a mean diameter of 0.15-0.2 mm held at a temperature near the melting point of the particles and at a pressure sufficient to cause sintering of the particles to fuse them together and form a uniform structure.

Although the membrane 202 and the support member 404 are depicted as circular, this is illustrative only. In other embodiments, the membrane 202 and/or the support member 404 may be shaped as a square, a rectangle, an oval, etc. In general, the shape and the surface area of the support member, if it is used, is selected such that the surface of the support member is approximately the same size as or slightly smaller than the membrane disposed thereon.

The membrane 202 is disposed in contact with the substantially flat, smooth surface 406 of the support member 404 before the sample fluid is poured onto the membrane 202. The generally flat surface 406 helps keep the membrane 202 substantially flat after the sample fluid is drained. The fluid permeable solid support 404 can also serve as a reservoir for fluid passed through the membrane 202 and the fluid permeable solid support 404, to provide the additional benefit of preventing the membrane 202 and viable cells disposed thereon from drying out during the detection process. Drying can be detrimental to the viability of the cells retained on the membrane 202.

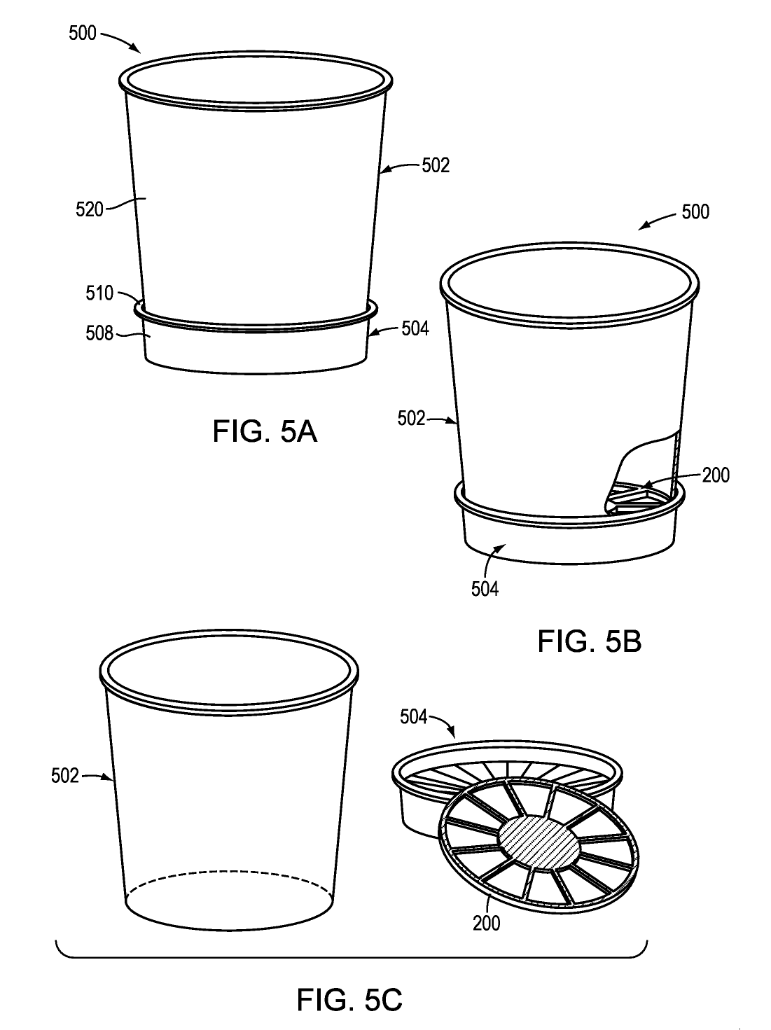

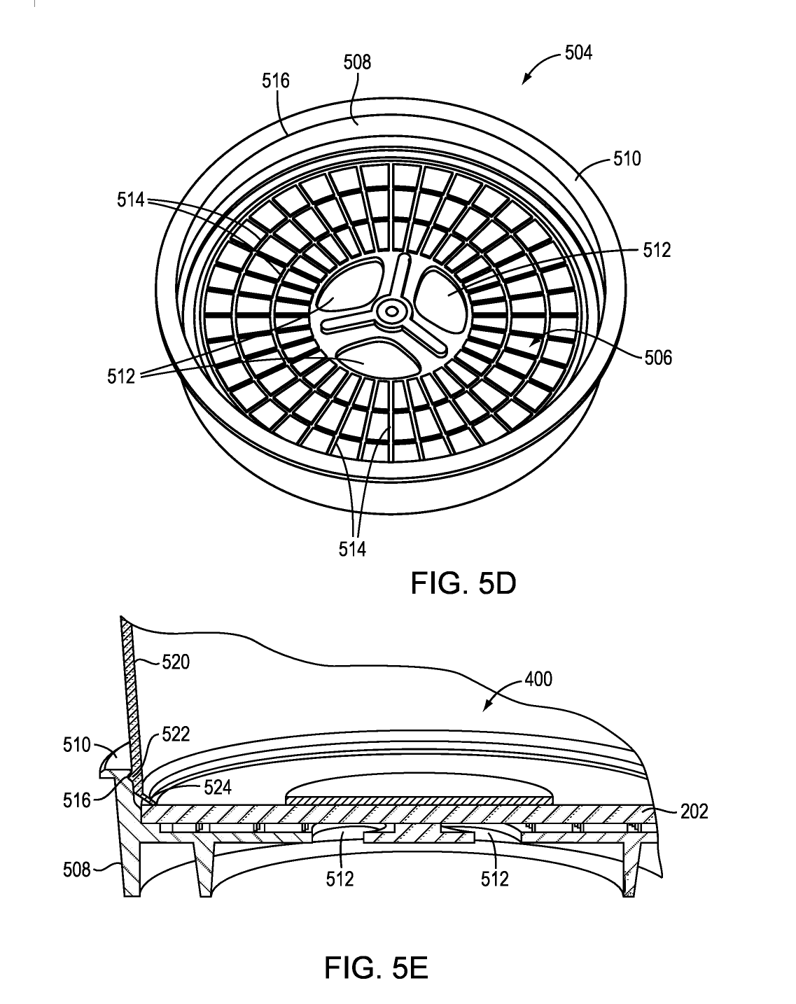

With reference to FIGS. 5A-5E, a cup and base assembly 500 having a cup 502 and a base 504 is used to facilitate the capture of cells present in a liquid sample on a membrane (e.g., the membrane 202) disposed within the base 504. The base 504 has a surface 506 (see, FIG. 5D), an outer wall 508, and a lip 510. The surface 506 defines at least one opening 512 and, optionally, circular and radial protrusions or grooves 514 to facilitate drainage of liquid passed through the membrane 202. The wall 508 has a circumferential groove 516 under the lip 510. In certain embodiments (see, FIG. 5D), the cup 502 comprises a wall 520 having a circumferential protrusion 522 adapted to mate with the base groove 516 to releasably interlock the cup 502 to the base 504. A lip section 524 of the wall 520, i.e., the section below the protrusion 522, inclines inwardly to form a circumferential sealing lip adapted to contact an upper surface of the porous membrane 202. The lip section 524 also captures the porous membrane 202 (and in certain embodiments the frame 200 and/or the support member 404) between the cup 520 and the base 504.

More generally, a membrane and any components for holding the membrane generally flat, such as a holder having spokes (described with reference to FIGS. 2A and 2B), masks (described with reference to FIGS. 3A and 3B), and/or the supporting member (described with reference to FIGS. 4A and 4B) can be received within the cup and base assembly 500 and disposed on the surface 506 of the base 504. The cup 502 then is disposed over the membrane assembly such that the wall protrusion 522 fits into the groove 516 of the base 504, as depicted in FIG. 5E. This fit helps ensure the proper positioning between the cup 502 and the base 504, particularly with respect to the membrane 202 contained therebetween. The dimensions of the section 524 (e.g., the length, the angle of inclination, etc.) are selected such that the section 524 presses against the membrane assembly 400 disposed in the base 504 to provide a fluidic seal and ensure a flat membrane 202.

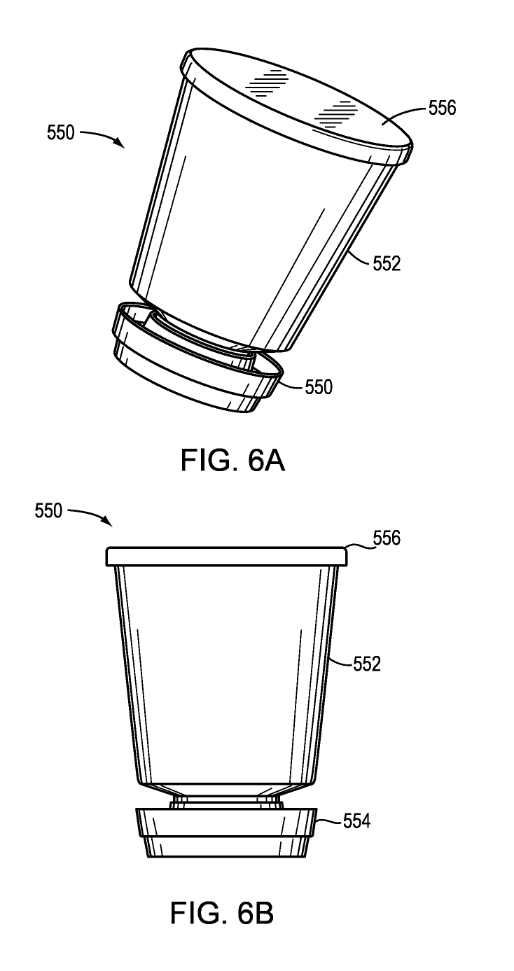

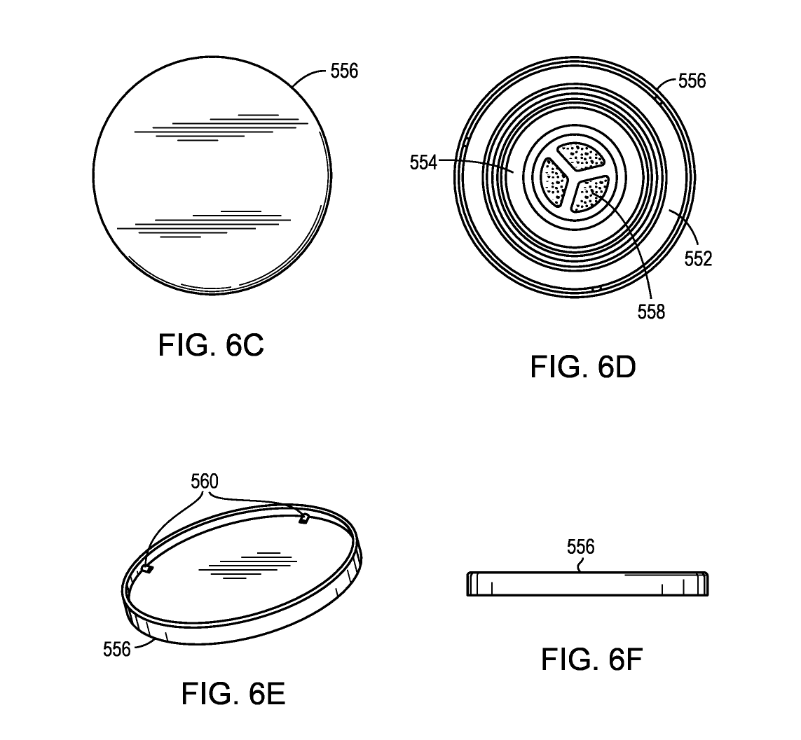

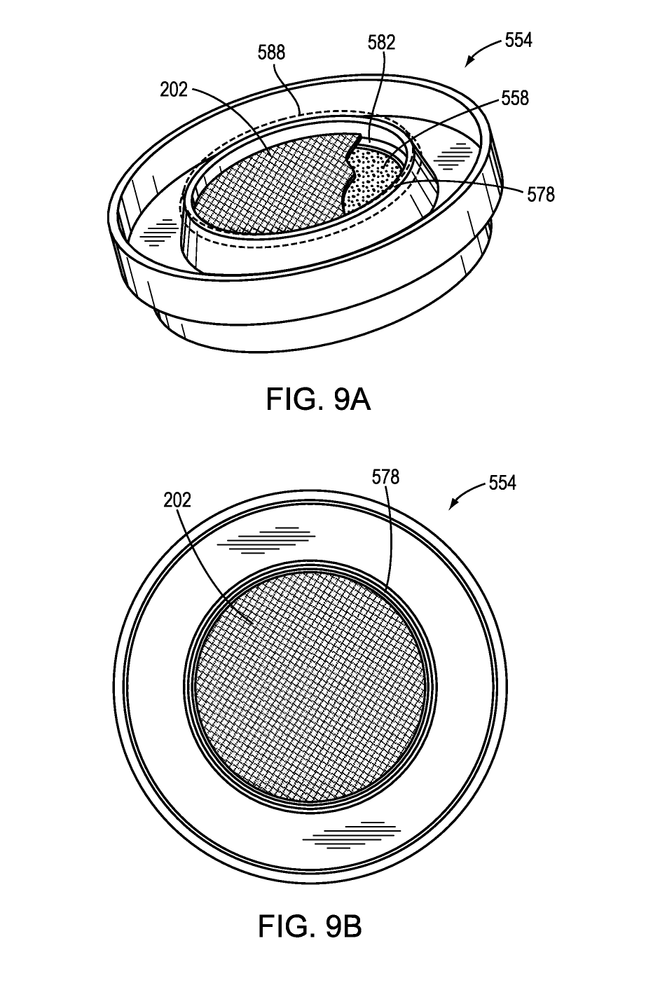

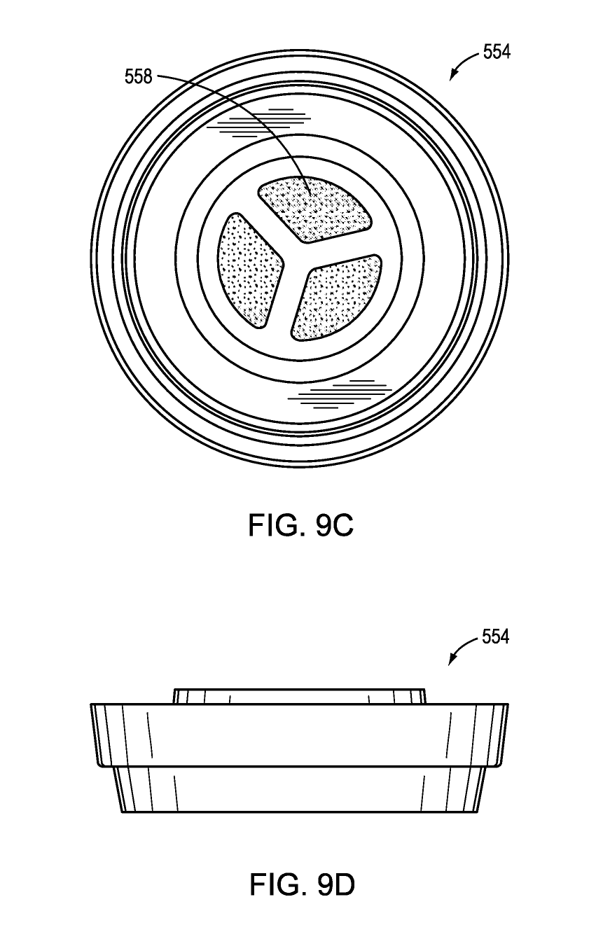

FIGS. 6A-6D depict another embodiment of a cup and base assembly 550. The cup and base assembly 550 has a cup 552 and a base 554 that in many aspects function similarly to the cup 502 and the base 504. The cup and base assembly 550 may also optionally contain a lid 556 for keeping the interior of the cup 552 protected from contaminants, both before and after use. A support member 558 (such as the support 404) is disposed in the base 554 for supporting the membrane 202 (depicted in FIGS. 9A and 9B). In the embodiment depicted, the lid 556 is substantially circular to interfit with cup 552, although any complementary shapes would be suitable. The lid 556 is shown in greater detail in FIGS. 6E and 6F, including ridges 560 that provide a small offset between the top of the cup 552 and a bottom surface of the top of the lid 556.

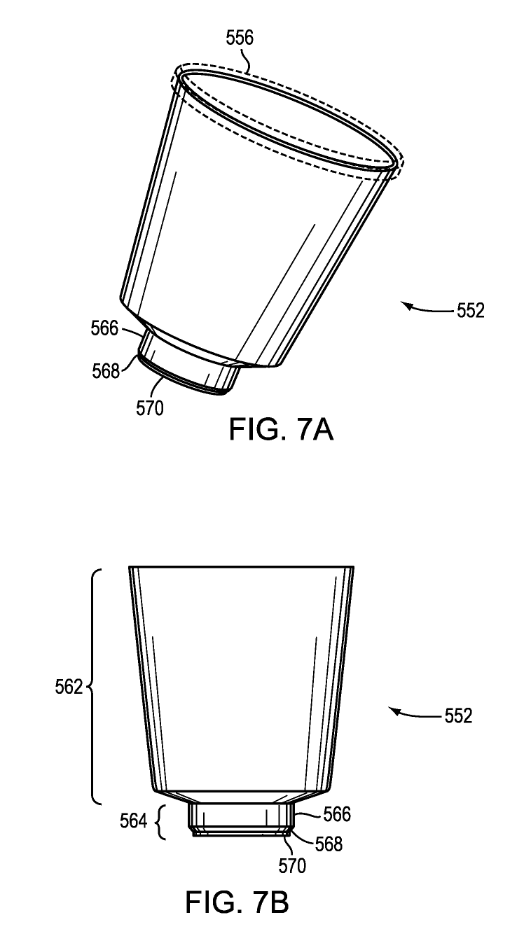

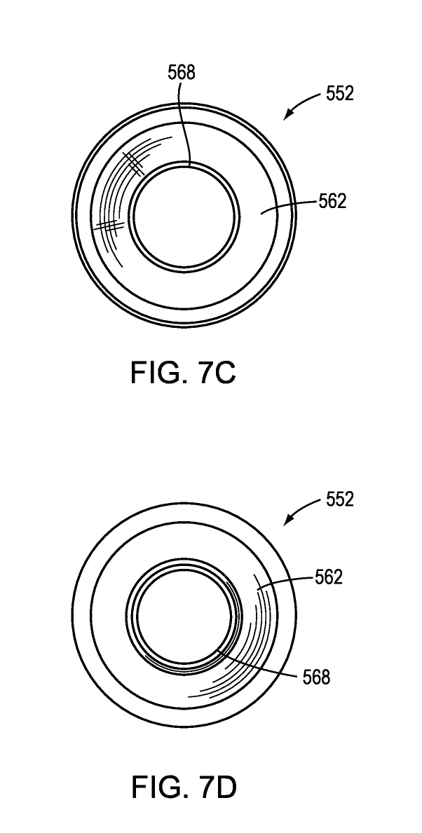

FIGS. 7A-7D depict the cup 552 in greater detail. The cup 552 includes an upper portion 562 that is substantially hollow and tapers out towards the top to provide an increased sectional area into which fluid may be poured. Further tapering directs the fluid toward a lower section 564 that is adapted to be received within the base 554. A vertical segment 566 can provide increased stability when the cup 552 is disposed within the base 554, from which a lip section 568 (similar to lip section 524) extends at an angle. A further vertical section 570 may also be provided for contacting the membrane 202.

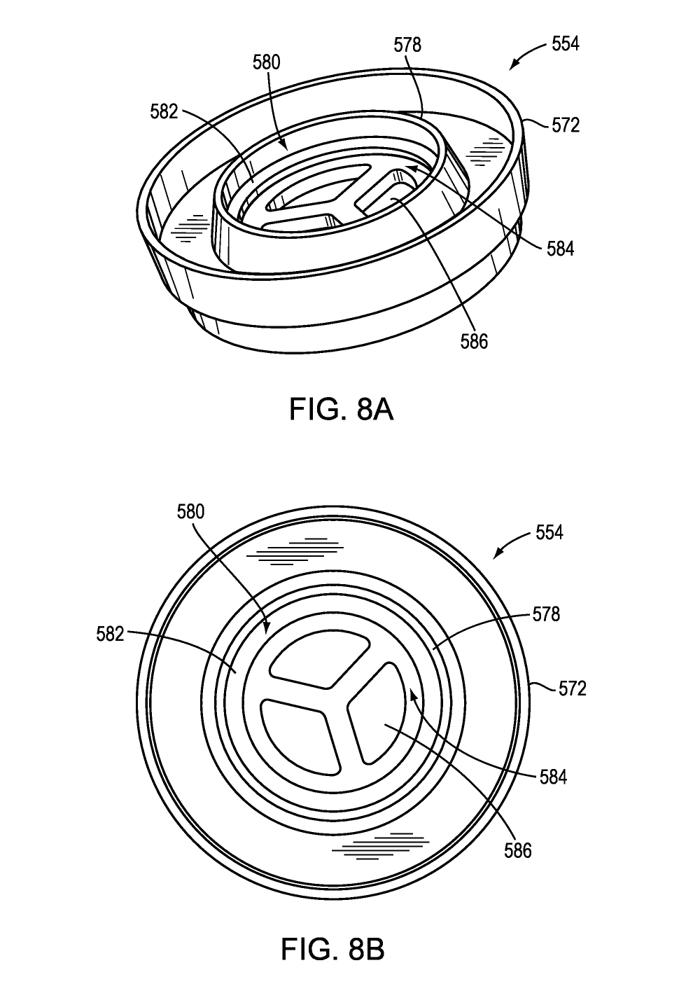

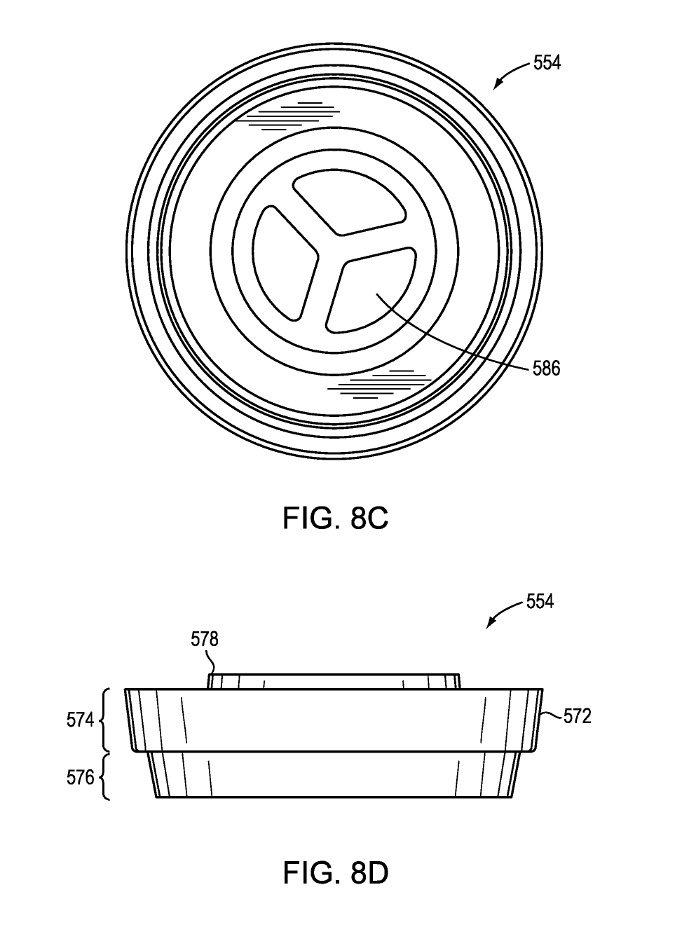

FIGS. 8A-8D depict the base 554. The base 554 includes an outer wall 572 defining an upper portion 574 that may catch extraneous fluid. A lower portion 576 is adapted to be received within a stage (described in detail below), and may be tapered to provide a tight fit when mounted thereon. An interior wall 578 defines a central recess 580 for receiving the cup 552, and more particularly the vertical segment 566. A tight fit and overlap between the vertical segment 566 and the interior wall 578 help ensure a stable fit while the cup 552 is mounted on the base 554. A ledge 582 for receiving the membrane 202 is located at a bottom of the interior wall 578, and further defines a recess 584 in the middle to receive the support member 558. The relationship of the base 554, the membrane 202, and the support member 558 is depicted in FIGS. 9A-9D, along with an optional lid 588 (depicted transparently in FIG. 9A). Openings 586 may be provided in the bottom of the base 554, similar to the openings 512.

In certain embodiments, the cell capture system, in particular the porous membrane, has a sterility assurance level less than 10⁻⁶, 10⁻⁷, 10⁻⁸, or 10⁻⁹. This can be achieved, for example, by sterilizing the cell capture system, via techniques known in the art, for example, via autoclaving, exposure to ionizing radiation, for example, gamma radiation or exposure to a sterilizing fluid or gas, for example, ethylene oxide or vaporized hydrogen peroxide. The cell capture system can be enclosed within a receptacle (e.g., a bag), prior to, during, or after sterilization. The cell capture system can be placed within a receptacle (e.g., a bag) and sealed (e.g., hermetically sealed) before terminal sterilization (e.g., via exposure to ionizing radiation).

In another embodiment, the invention provides a cell capture cup comprising an open cylindrical portion and an annular seal adapted to mate with a base comprising the cell capture system of any one of the foregoing aspects and embodiments. The cell capture cup and base can have a sterility assurance level less than 10⁻⁶, 10⁻⁷, 10⁻⁸, or 10⁻⁹, which can be achieved using any or all of the approaches discussed herein.

### (II) Cell Capture Method

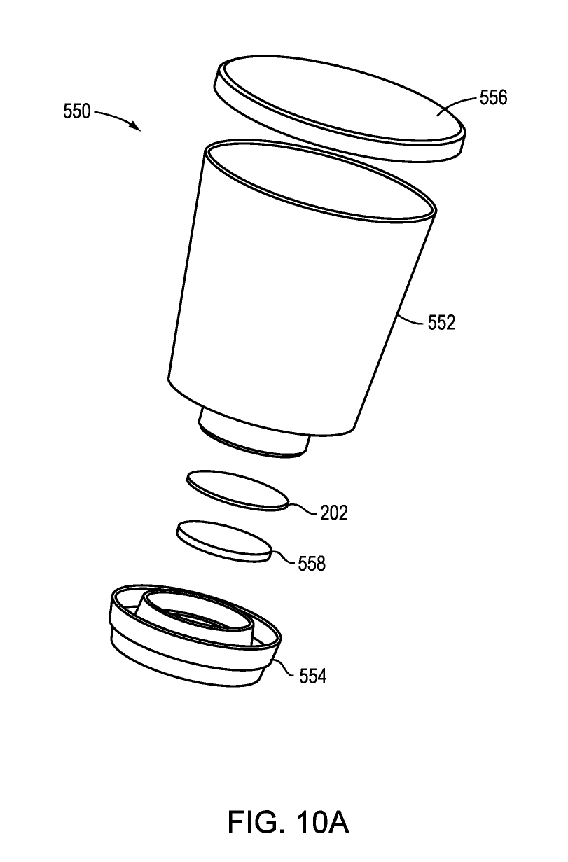

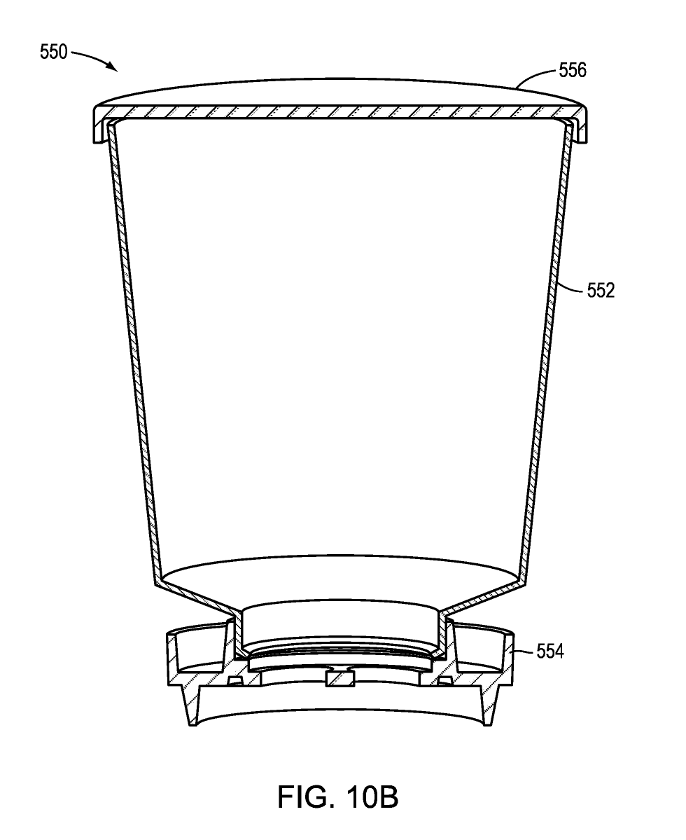

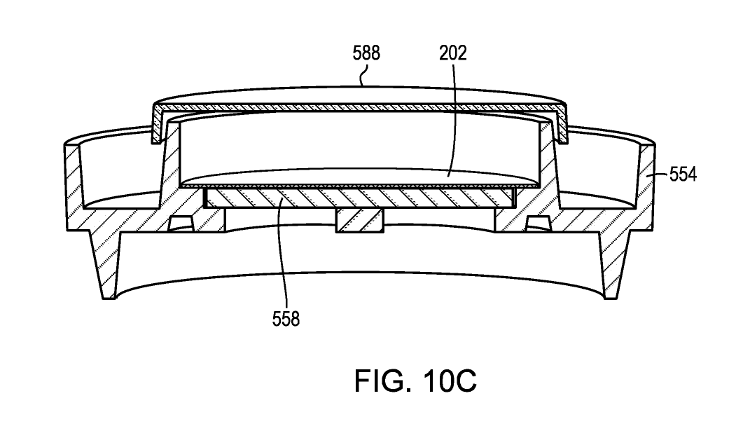

FIG. 10A depicts the components of an exemplary cup and base assembly 550. The porous support member 558 and the membrane 202 are disposed in the center of the base 554. The cup 552 then is installed on top of the membrane 202, helping to maintain the membrane 202 in a flat position. The lid 556 may be provided on top of the cup 552 to protect the interior of the cup 552 from being contaminated. FIG. 10B depicts the fitting of the components without the membrane 202 and the support 558.

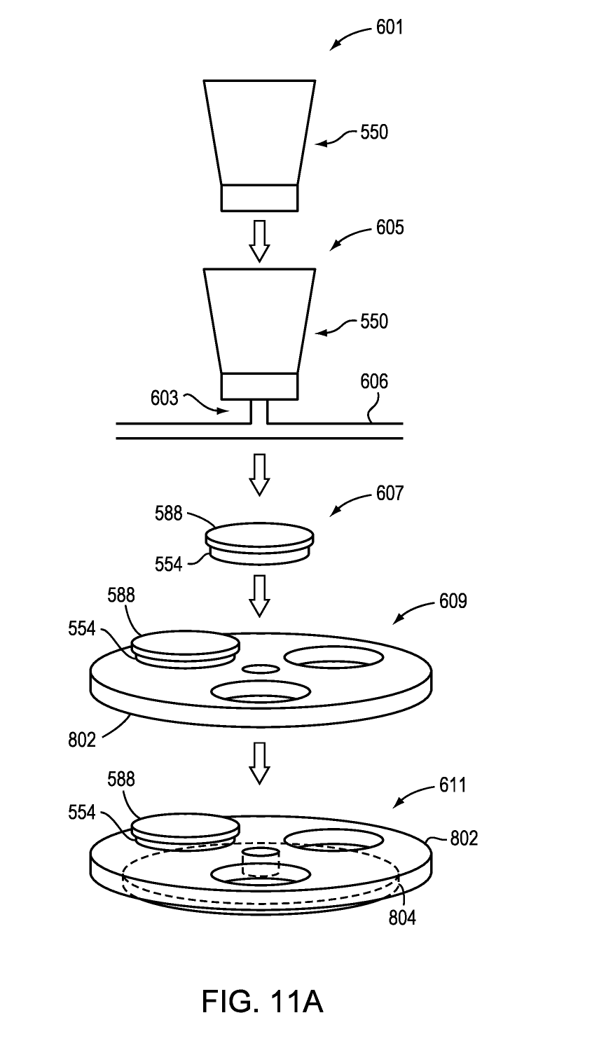

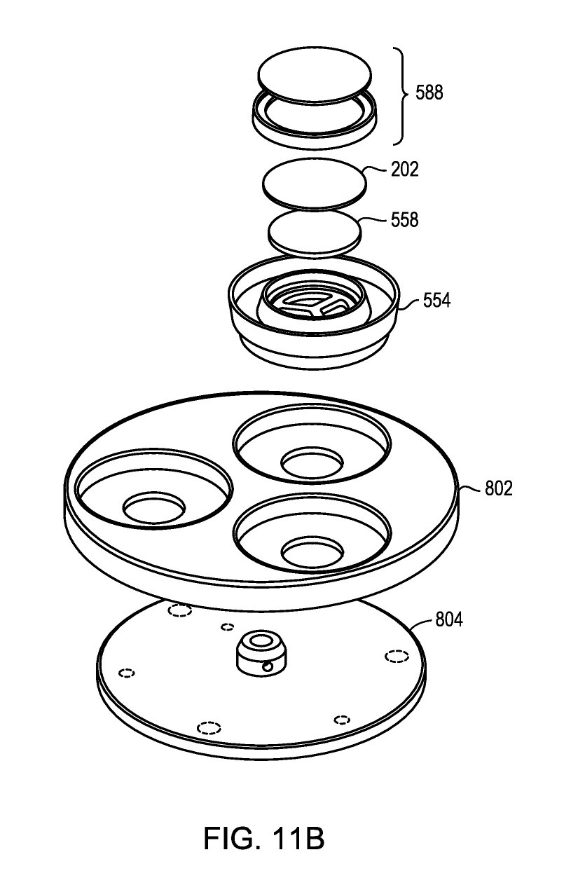

During use, a sample fluid is poured into the cup 552. Due to the tapers of the cup 552, the fluid wets the membrane assembly and passes through the membrane 202. The fluid typically passes through the membrane assembly (e.g., through the membrane 202, and the porous support member 558, if one is used) toward the base 554. Negative pressure, for example, a vacuum, can be advantageously employed to draw fluid through the membrane 202 to the openings 586 (e.g., in the embodiment of FIG. 5E, via the grooves 514), and to help keep the membrane substantially flat. After the fluid is drawn through the cup and base assembly 550, any particles and/or cells in the fluid that cannot pass through membrane 202 are retained on the upper exposed surface of the membrane 202. After pouring the fluid into the cup assembly, the cup 552 may be separated from the base 554, as depicted in FIG. 10C, and a lid 558 placed on top of the base 554. The lid 588 may be provided on top of the base 554 to protect the moistened membrane 202 and support 558 from contamination when the base is transferred to the stage 802 (FIG. 11B) or when the base containing membrane 202 is incubated, for example, from 15 minutes to 8 hours, from 30 minutes to 6 hours, or from 30 minutes to 3 hours, to permit the captured viable cells to proliferate.

An exemplary flow chart showing the assembly of the cell capture system, the passage of liquid sample through the cell capture system and the assembly of the membrane holder for use in an exemplary optical detection system is shown in FIG. 11A.

With reference to FIG. 11A, in step 601, a cup and base assembly 550 is provided. In step 603, the cup and base assembly 550 is coupled to a vacuum system (e.g., a vacuum manifold 606) and a negative pressure is applied to the underside of the cup and base assembly 550. In step 605, the liquid sample is poured into the cup and base assembly 550, and any cells present in the liquid sample are retained on the upper exposed surface of the porous membrane 202. This pouring step can occur before, at the same time, or after step 603. It is contemplated that the substantially non-autofluorescent membrane permits a flow rate therethrough of at least 5 or at least 10 mL/cm²/min with a vacuum of about 5 Torr or about 10 Torr. The cells can then be stained with a viability stain or a viability staining system, for example, as discussed in Section III so that it is possible to selectively detect and distinguish viable cells from non-viable cells. The cells may optionally be washed with a physiologically acceptable salt and/or buffer solution to remove residual non-specifically bound fluorescent dye and/or quencher.

In step 607, the membrane assembly is removed from the cup 552, typically in combination with the base 554, though removal independent from the base 554 may be possible. In step 609, the base 554 (and thereby the membrane 202) is disposed on a stage 802. In step 611, the stage 802 is disposed on a chuck 804. The stage 802 and the chuck 804 are described in greater detail below. Steps 609 and 611 may be performed in reverse order or concurrently. The stage 802 and the chuck 804 can be located in the exemplary detection system 100 of FIG. 1A at the start of the process in order to detect any cells (viable and/or non-viable cells) and/or particles captured on the surface of membrane 202. In other embodiments, the stage 802 and/or the chuck 804 may be assembled with the base 554 remote from the detection system 100.

Furthermore, the cells can be captured on a planar membrane using the cup assembly described in U.S. Patent Application Ser. No. 61/899,436 (Atty. Docket No. CHR-038PR) and International Application Serial No. PCT/US2014/063950 (Atty. Docket No. CHR-038PC).

### (III) Cell Staining

Despite the cell viability stains available, there is a need for specific dyes that can be used to selectively detect viable cells (both single (individual) cells and clusters of cells) with little or no background fluorescence emanating from non-viable cells, especially under the conditions (for example, using the excitation light) used to excite the viable cells using the detection system described herein. In addition, the stains should not compromise the viability of the cells being detected. Furthermore, if desired, the stains should permit the simultaneous proliferation and staining of viable cells in the cell sample.

The present invention is based, in part, upon the discovery of certain fluorescent dyes (which are in the form of substantially non-fluorescent precursors) that are particularly effective as viability stains in the detection methods described herein. In particular, once the cells are captured on the permeable membrane, the cells can be stained using a dye precursor selected from the group consisting of a bioactivatable tetrazolium dye and an esterase-activatable dye. The dyes used herein are particularly effective for the detection of individual viable cells because of the their brightness once excited relative to background. The dye precursor, prior to conversion, is substantially non-fluorescent, i.e., the dye precursor emits less than 20%, 10%, 5%, or 1% of the fluorescence emitted from the fluorescent dye when excited by light at or about the λₘₐₓ of absorption of the fluorescent dye. As a result, the use of the dyes permits the detection of individual viable cells relative to non-viable cells. As used herein, the term “non-viable cells” is understood to mean cells that are already dead or cells undergoing cell death.

In one embodiment, the bioactivatable tetrazolium dye is a tetrazolium-containing compound that undergoes conversion to a fluorescent label in a viable cell. In certain embodiments, the bioactivatable tetrazolium dye is reduced by a viable cell to produce a fluorescent label. Preferably, the bioactivatable tetrazolium dye is substantially non-fluorescent until reduced to a fluorescent label by the viable cell. Further description of exemplary bioactivatable tetrazolium dyes is provided below.

In another embodiment, the esterase-activatable dye is an ester-containing compound that becomes activated by an esterase enzyme within a cell to produce a fluorescent label. In certain embodiments, the esterase-activatable dye is an ester-containing compound that undergoes cleavage of one or more ester bonds by an esterase enzyme to produce a fluorescent label. Preferably, the esterase-activatable dye is substantially non-fluorescent until it is converted to a fluorescent label by an esterase enzyme in a viable cell. Further description of exemplary esterase-activatable dyes is provided below.

The particular staining protocol used for each dye will depend upon a variety of factors, such as, the cells being detected, and whether the cells are going to be stained and detected immediately or whether the cells are going to be cultured for a period of time, for example, from 30 minutes to several hours, to permit the cells to proliferate so that a plurality of cells rather than a single cell is detected at a particular locus. Exemplary staining and, where desired, culturing protocols are discussed in the following sections. Furthermore, given that the dye precursors are converted into fluorescent dyes by metabolic activity within the cells, the incubation of the cells prior to staining can enhance metabolic activity within the cells prior to staining and detection.

Cells exposed to the dye precursor may be incubated for a length of time sufficient so that a viable cell, if present, produces fluorescent label from one or both of the bioactivatable tetrazolium dye and an esterase-activatable dye. In certain embodiments, the incubating is performed for a least about 1 second, 10 seconds, 1 minute, 5 minutes, 10 minutes, 15 minutes, 20 minutes, 25 minutes, 30 minutes, or 45 minutes. In certain embodiments, the incubating is performed for a period of time that does not exceed about 5 seconds, 10 seconds, 1 minute, 5 minutes, 10 minutes, 15 minutes, 20 minutes, 25 minutes, 30 minutes, or 45 minutes.

In certain embodiments, an oxidant that is impermeable to the cell membrane of a viable cell may be used to prevent or reduce the occurrence of premature conversion (e.g., through chemical reduction) of the bioactivatable tetrazolium dye to a fluorescent dye. In particular, the oxidant desirably prevents or reduces the occurrence of conversion (e.g., through chemical reduction) of the bioactivatable tetrazolium dye to a fluorescent dye before the bioactivatable tetrazolium dye enters a viable cell. The oxidant may be applied to the cell sample prior to exposing the cells to a bioactivatable tetrazolium dye, concurrently with exposing the cells to a bioactivatable tetrazolium dye, or after exposing the cells to a bioactivatable tetrazolium dye. In certain embodiments, the oxidant is mixed with the bioactivatable tetrazolium dye to form a mixture that is applied to cells in the cell sample. Desirably, the oxidant is applied to the cell sample in an amount sufficient so that no more than 1% (w/w), 5% (w/w), 10% (w/w), 15% (w/w), 20% (w/w), or 30% (w/w) of the bioactivatable tetrazolium dye originally applied to the cell sample undergoes conversion to a fluorescent dye outside a viable cell. Potassium ferricyanide is an exemplary oxidant that is impermeable to the cell membrane of a viable cell. The use of such an oxidant can be particularly beneficial when the cell sample or testing apparatus contains a material (e.g., a gold metal surface) that can facilitate conversion (e.g., through chemical reduction) of the bioactivatable tetrazolium dye to a fluorescent dye outside of a viable cell.

A. Bioactivatable Tetrazolium Dye

The bioactivatable tetrazolium dye is a tetrazolium-containing compound that undergoes conversion to a fluorescent label in a viable cell. Tetrazolium is a chemical group having the following structure:

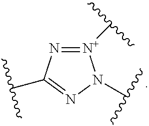 [SMILES: *c1nn(*)[n+](*)n1]

Preferably, the bioactivatable tetrazolium dye does not possess significant fluorescence until reduced to a fluorescent label by the viable cell.

One exemplary collection of bioactivatable tetrazolium dyes is represented by Formula I:

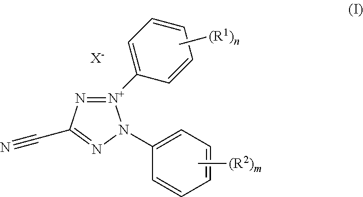

wherein:
X is halogen or ⁻ OC(O)R³;
R¹ and R² each represent independently for each occurrence hydrogen, C₁-C₆ alkyl, C₃-C₆ cycloalkyl, C₁-C₆ alkylene-(C₃-C₆ cycloalkyl), halogen, C₁-C₆ haloalkyl, hydroxyl, C₁-C₆ alkoxyl, —O—(C₃-C₆ cycloalkyl), nitro, cyano, —C(O)R³, —CO₂R³, —C(O)N(R⁴)₂, or —N(R⁴)C(O)R³;
R³ represents independently for each occurrence C₁-C₆ alkyl, C₁-C₆ haloalkyl, C₃-C₆ cycloalkyl, C₁-C₆ alkylene-(C₃-C₆ cycloalkyl), aryl, or heteroaryl;
R⁴ represents independently for each occurrence hydrogen, C₁-C₆ alkyl, C₃-C₆ cycloalkyl, C₁-C₆ alkylene-(C₃-C₆ cycloalkyl), or two occurrences of R⁴ attached to the same nitrogen atom are taken together with the nitrogen atom to which they are attached to form a 3-7 membered heterocyclic ring; and
m and n each represent independently 1, 2, or 3.

In certain embodiments, R¹ and R² each represent independently for each occurrence hydrogen, C₁-C₆ alkyl, or C₃-C₆ cycloalkyl. In certain embodiments, m and n are 1.

The description above describes multiple embodiments relating to compounds of Formula I. The patent application specifically contemplates all combinations of the embodiments.

In certain other embodiments, the bioactivatable tetrazolium dye is represented by Formula I-A:

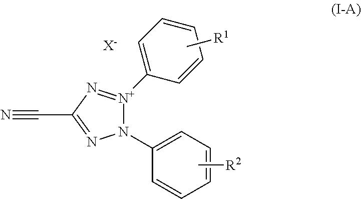

wherein: X is halogen; and R¹ and R² each represent independently C₁-C₆ alkyl, C₃-C₆ cycloalkyl, or C₁-C₆ haloalkyl.

In certain embodiments, in connection with Formula I-A, R¹ and R² each represent independently C₁-C₆ alkyl, such as methyl (which may be located at the para-position of the phenyl rings).

The description above describes multiple embodiments relating to compounds of Formula I-A. The patent application specifically contemplates all combinations of the embodiments.

In certain embodiments, the bioactivatable tetrazolium dye is 5-cyano-2,3-di-(p-tolyl)tetrazolium halide. In certain other embodiments, the bioactivatable tetrazolium dye is 5-cyano-2,3-di-(p-tolyl)tetrazolium chloride.

B. Esterase-Activatable Dye

The esterase-activatable dye is an ester-containing compound that becomes activated by an esterase enzyme to produce a fluorescent label. Ester is a chemical group having the following structure: R*—CO₂—R**, where R* and R** are independently a carbon fragment, such as alkyl, aryl, or aralkyl. Preferably, the esterase-activatable dye does not possess significant fluorescence until converted to a fluorescent label by an esterase enzyme in a viable cell.

In certain embodiments, the esterase-activatable dye is represented by Formula II:

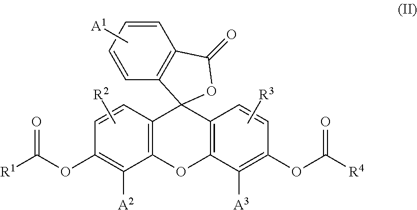

or a salt thereof, wherein:
R¹ and R⁴ each represent independently C₁-C₆ alkyl, C₃-C₆ cycloalkyl, C₁-C₆ alkylene-(C₃-C₆ cycloalkyl), C₁-C₆ haloalkyl, aryl, or heteroaryl;
R² and R³ each represent independently hydrogen, C₁-C₆ alkyl, C₃-C₆ cycloalkyl, C₁-C₆ alkylene-(C₃-C₆ cycloalkyl), halogen, C₁-C₆ haloalkyl, hydroxyl, or C₁-C₆ alkoxyl;
R⁵ represents independently for each occurrence hydrogen, C₁-C₆ alkyl, or C₃-C₆ cycloalkyl;
A¹ is hydrogen,

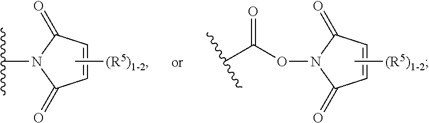

and
A² and A³ each represent independently hydrogen, C₁-C₆ alkyl, C₃-C₆ cycloalkyl, C₁-C₆ alkylene-(C₃-C₆ cycloalkyl), C₁-C₆ haloalkyl, or C₁-C₆ alkylene-N[—C₁-C₄ alkylene-CO₂—C₁-C₄ alkylene-O—C(O)—C₁-C₄ alkyl]₂.

In certain embodiments, R¹ and R⁴ are C₁-C₆ alkyl. In certain other embodiments, R¹ and R⁴ are methyl.

In certain embodiments, R² and R³ are hydrogen.

In certain embodiments, A¹ is

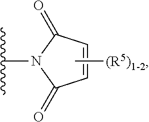

and A² and A³ are hydrogen. In certain other embodiments, A¹ is

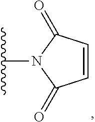 [SMILES: *N1C(=O)C=CC1=O]

and A² and A³ are hydrogen. In certain other embodiments, A¹ is

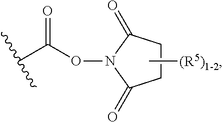

and A² and A³ are hydrogen. In certain other embodiments, A¹ is

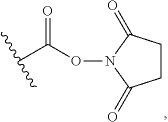 [SMILES: *C(=O)ON1C(=O)CCC1=O]

and A² and A³ are hydrogen. In certain other embodiments, A¹ is hydrogen, and A² and A³ are C₁-C₆ alkylene-N[—C₁-C₄ alkylene-CO₂—C₁-C₄ alkylene-O—C(O)—C₁-C₄ alkyl]₂. In certain other embodiments, A¹ is hydrogen, and A² and A³ are —CH₂—N[—CH₂—CO₂—CH₂—O—C(O)—CH₃]₂.

The description above describes multiple embodiments relating to compounds of Formula II. The patent application specifically contemplates all combinations of the embodiments.

Exemplary esterase-activatable dyes include, for example, fluorescein diacetate 5-maleimide, Calcein-AM, 5-carboxyl-fluorescein diacetate N-succinimidyl ester, Calcein Blue AM, Carboxycalcein Blue AM, fluorescein diacetate, carboxyfluorescein diacetate, 5-carboxyfluoresein diacetate AM, sulfofluorescein diacetate, and BCECF-AM. See, for example, U.S. Pat. No. 5,534,416, for further discussion of exemplary esterase-activatable dyes, which is hereby incorporated by reference.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic ring systems having two or more carbocyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, and/or aryls. In certain embodiments, the aromatic ring is substituted at one or more ring positions with halogen, alkyl, hydroxyl, or alkoxyl. In certain other embodiments, the aromatic ring is not substituted, i.e., it is unsubstituted.

The term “aralkyl” refers to an alkyl group substituted with an aryl group.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted.

The terms “heterocyclyl” and “heterocyclic group” are art-recognized and refer to saturated or partially unsaturated 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isoxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. In certain embodiments, the heterocyclcyl group is not substituted, i.e., it is unsubstituted.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Various compounds described herein may also be in the form of a salt, such as a pharmaceutically acceptable salt. As used herein, the term “pharmaceutically acceptable salt” refers to any pharmaceutically acceptable salt (e.g., acid or base) of a compound of the present invention which, upon administration to a viable cell, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

As a general matter, if a variable is not accompanied by a definition, then the previous definition of the variable controls.

In certain embodiments, the fluorescent dye precursor is used at a concentration in the range of from about 0.1 μM to about 50 μM, from about 0.5 μM to about 30 μM, or from about 1 μM to about 10 μM, when applied to cells.

In certain embodiments, the non-viable cells emit no or substantially no fluorescence detectable by the detector upon exposure to the light. In certain other embodiments, the non-viable cells emit no or substantially no fluorescence upon exposure to the beam of light.

The detection method can be performed on single cells, clusters of cells or colonies of cells. Under certain circumstances, for example, to increase the sensitivity of the assay, it may be desirable to culture the cells under conditions that permit cell proliferation prior to and/or during and/or after exposing the cells to the fluorescent dye precursor. The culture conditions, including, the choice of the growth media, the temperature, the duration of the culture, can be selected to permit at least one of cells in the sample to have one or more cell divisions.

For example, depending upon the sensitivity required, the cells, once captured on the membrane, can be contacted with growth media and/or spore germination initiators and then permitted to proliferate for one or more doubling times to increase the number of cells at a particular locus on the membrane.

In one embodiment, the cells are captured on membrane 202, a solution containing the fluorescent dye precursor and growth medium (e.g., Nutrient Broth T7 105 from PML Microbiologicals, Wisonville, Oreg.) are poured into the cup assembly and pulled through membrane 202 via vacuum suction. The lid 588 is then placed upon base 554 (see, FIG. 10C), and the resulting unit can be placed in an incubator at a preselected temperature (e.g., 32° C. or 37° C.) for a desired length of time (e.g., from 15 minutes to 8 hours, or from 30 minutes to 4 hours) depending upon the doubling time of the organisms. During this time, the membrane 202 remains moist in view of the growth media and stain present within solid support 558. This approach also provides more time for the fluorescent dye precursor to permeate the cells and then be converted into the fluorescent dye so as to fluorescently stain the cells. After incubation, the base 554 can then be transferred to and placed into stage 802 for insertion into the detection device.

In another embodiment, porous membrane 202 having cells and/or microcolonies disposed thereon can be placed upon growth media containing the dye precursor and/or having the dye precursor disposed upon a surface adjacent the porous membrane and incubated under conditions (for example, 32° C. or 37° C.) and for a time (for example, 15 minutes to 1 hour) sufficient to permit the dye precursor to gradually permeate the membrane and then gently permeate viable cells disposed upon the membrane. Thereafter, the dye precursor is converted into a fluorescent dye by metabolic activity within the viable cells. This approach maintains the integrity of the colonies, which may otherwise be disturbed or destroyed when a dye precursor is applied directly to the colonies. This is especially important when colonies or microcolonies are being detected as this step may result in disposal of the cells or clusters of cells that may then be erroneously counted as additional cells or clusters of cells.

In certain embodiments, the beam of light used to excite the fluorescent dye or fluorescent dyes has a wavelength in the range of from about to 350 nm to about 1000 nm, from about 350 nm to about 900 nm, from about 350 nm to about 800 nm, from about 350 nm to about 700 nm, or from about 350 nm to about 600 nm. For example, the wavelength of excitation light is at least in one range from about 350 nm to about 500 nm, from about 350 nm to about 550 nm, from about 350 nm to about 600 nm, from about 400 nm to about 550 nm, from about 400 nm to about 600 nm, from about 400 nm to about 650 nm, from about 450 nm to about 600 nm, from about 450 nm to about 650 nm, from about 450 nm to about 700 nm, from about 500 nm to about 650 nm, from about 500 nm to about 700 nm, from about 500 nm to about 750 nm, from about 550 nm to about 700 nm, from about 550 nm to about 750 nm, from about 550 nm to about 800 nm, from about 600 nm to about 750 nm, from about 600 nm to about 800 nm, from about 600 nm to about 850 nm, from about 650 nm to about 800 nm, from about 650 nm to about 850 nm, from about 650 nm to about 900 nm, from about 700 nm to about 850 nm, from about 700 nm to about 900 nm, from about 700 nm to about 950 nm, from about 750 to about 900 nm, from about 750 to about 950 nm or from about 750 to about 1000 nm. Certain ranges include from about 350 nm to about 600 nm and from out 600 nm to about 750 nm.

In certain embodiments, the fluorescent dye can be excited to undergo fluorescence by radiation from a red laser, for example, a red laser that emits light having a wavelength in the range of 620 nm to 640 nm.

The fluorescent emission can be detected within a range of from about 350 nm to about 1000 nm, from about 350 nm to about 900 nm, from about 350 nm to about 800 nm, from about 350 nm to about 700 nm, or from about 350 nm to about 600 nm. For example, the fluorescent emission can be detected within a range from about 350 nm to 550 nm, from about 450 nm to about 650 nm, from about 550 nm to about 750 nm, from about 650 nm to about 850 nm, or from about 750 nm to about 950 nm, from about 350 nm to about 450 nm, from about 450 nm to about 550 nm, from about 550 nm to about 650 nm, from about 650 nm to about 750 nm, from about 750 nm to about 850 nm, from about 850 nm to about 950 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to 700 nm, from about 700 nm to about 750 nm, from about 750 nm to about 800 nm, from about 800 nm to about 850 nm, from about 850 nm to about 900 nm, from about 900 nm to about 950 nm, or from about 950 nm to about 1000 nm. In certain embodiments, the emitted light is detected in the range from about 660 nm to about 690 nm, from about 690 nm to about 720 nm, and/or from about 720 nm to about 850 nm. In certain embodiments, the emitted light is detected in the range from about 490 nm to about 540 nm and/or from about 590 nm to about 700 nm.

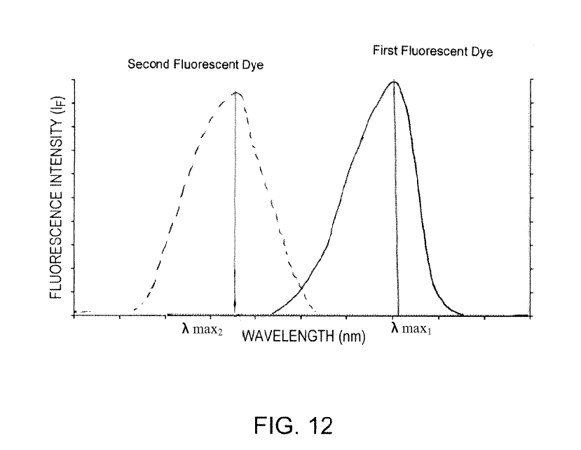

In each of the foregoing, the method can further comprise exposing the cells to a second, different membrane permeable fluorescent dye that labels viable cells, non-viable cells or a combination of viable and non-viable cells. FIG. 12 shows a schematic representation of the emission spectra of a first fluorescent dye and a second fluorescent dye illustrating the embodiment where the first fluorescent dye has a high-intensity emission at a wavelength substantially different from the high-intensity emission by the second fluorescent dye (e.g., the λₘₐₓ₁ of the emission spectrum of the first fluorescent dye is distinct from the λₘₐₓ₂ of the emission spectrum of the second fluorescent dye) so that the emission signals from both fluorescent dyes can be measured substantially independently.

### (IV) Cell Detection

Once the cell capture system has been used to capture cells originally present in the fluid sample, the membrane or the membrane assembly can be inserted into a membrane holder (e.g., holder 802) for insertion into a suitable detection system. Exemplary detection systems are described, for example, in International Patent Application No. PCT/IB2010/054965, filed Nov. 3, 2010, U.S. patent application Ser. No. 13/034,402, filed Feb. 24, 2011, International Patent Application No. PCT/IB2010/054966, filed Nov. 3, 2010, U.S. patent application Ser. No. 13/034,380, filed Feb. 24, 2011, International Patent Application No. PCT/IB2010/054967, filed Nov. 3, 2010, and U.S. patent application Ser. No. 13/034,515, filed Feb. 24, 2011. In the foregoing detection systems, a membrane is rotated while a beam of excitation light is directed onto the surface of the membrane. The emitted light is detected with at least one optical detector.

The invention provides a method of detecting the presence and/or quantity of viable cells in a liquid sample. The method comprises the steps of: (a) exposing viable cells, if any, retained by at least a portion of a substantially planar porous membrane after passing the liquid sample therethrough with a dye precursor selected from the group consisting of a bioactivatable tetrazolium dye and an esterase-activatable dye, where the dye precursor is converted to a fluorescent label by a viable cell; (b) scanning the portion of the porous membrane by rotating the porous membrane relative to a detection system comprising, (i) a light source emitting a beam of light of a wavelength adapted to excite the fluorescent label to produce an emission event, and (ii) at least one detector capable of detecting the emission event, thereby to interrogate a plurality of regions of the planar porous membrane and to detect emission events produced by excitation of fluorescent label associated with any viable cells; and (c) determining the presence and/or quantity of viable cells captured by the membrane based upon the emission events detected in step (b).

In order to facilitate rotation of the permeable membrane, the membrane can be disposed in a membrane holder. In one embodiment, for example, in a membrane assembly that comprises a mask and optional spokes, the membrane assembly may be inserted into a membrane holder that can be placed within sample assembly 120 of FIG. 1A. In particular, the membrane assembly maybe placed upon the rotating platform 130.

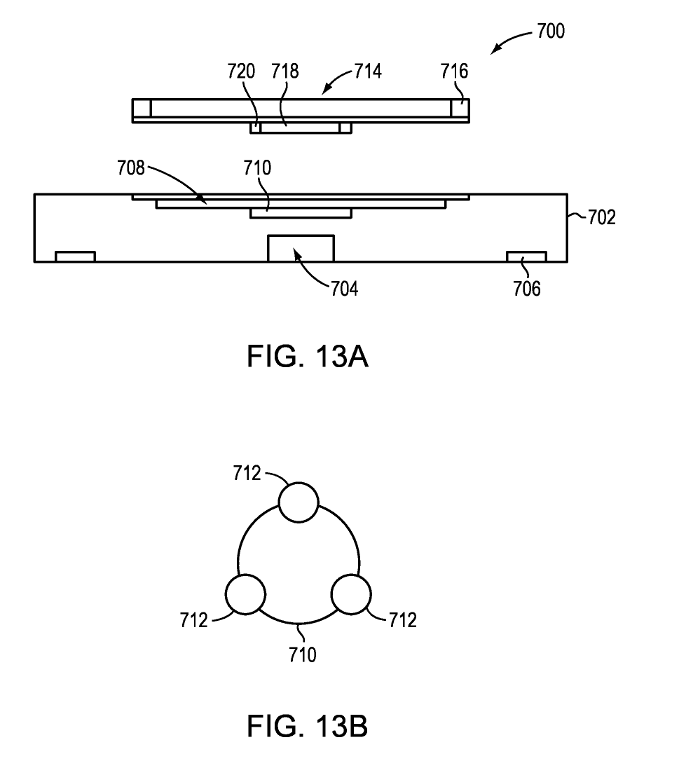

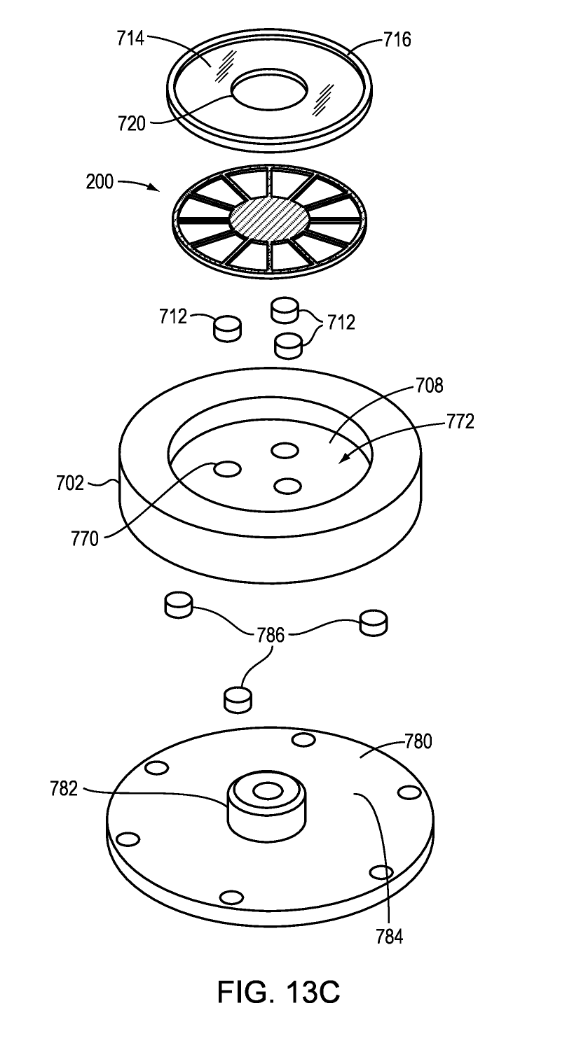

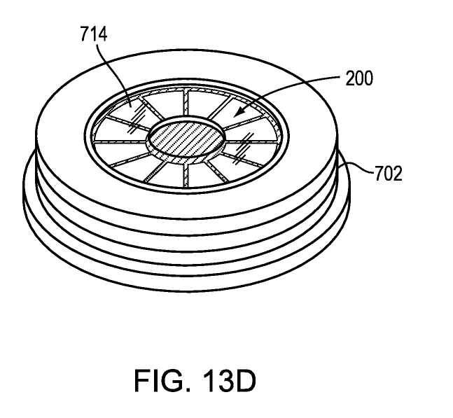

FIGS. 13A and 13B show an exemplary membrane holder that can be used in such a detection system. Membrane holder 700 comprises a container 702 (e.g., a metallic container made of aluminum) defining a central cylindrical recess 704 and an offset drive aperture or notch 706. The container 702 may be disposed upon a rotatable shaft such that the shaft is received within, coupled to, or otherwise engaged with the recess 704. The shaft may form a disk to support the holder 700 and can include a protrusion, such as a driver pin that couples with the drive notch 706. As a result, rotation of the disk about its axis of rotation correspondingly positively rotates the membrane holder 700 without slippage.

The container 702 also defines a chamber 708 to receive a membrane and any components for holding the membrane generally flat. These components include a holder having spokes (described with reference to FIGS. 2A and 2B), the masks (described with reference to FIGS. 3A and 3B), and/or or the porous supporting member (described with reference to FIGS. 4A-4B). Under the chamber 708, a plurality of magnets 712 can be disposed within the container 702. An exemplary magnet configuration 710 is depicted in FIG. 13B. The configuration 710 includes three magnets 712 located approximately in a circular pattern having a center at or near the axis of rotation of the container 702. The plane of the circle of magnets 712 is substantially parallel to the surface of the chamber 708. The container 702 further comprises a window 714 such as a glass, polycarbonate or perspex window enclosed in a magnetic ring 716. In certain embodiments, the magnets 712 are disk magnets and are used to maintain the elements in the chamber 708 during rotation (e.g., by attraction of the magnetic ring 716). It should be understood that the configuration 710 is for illustrative purposes only, and that other configurations, such as those having fewer or more than three magnets, may incorporate patterns other than a circular pattern, as well as other retention schemes, and are considered within the scope of the present invention.

The window 714 protects the underlying cell retaining membrane, as well as the cells, and can maintain the sterility of the membrane if is to be subsequently removed and incubated under conditions (e.g., temperature, moisture, and nutrition) to facilitate growth of the viable cells. The magnetic ring 716 can have an extension 718 forming a magnetic stainless steel ring 720. The center of the extension 718 is located at or near the axis of rotation of container 702. Accordingly, when the window 714 is disposed over a membrane assembly received in the chamber 708, the ring 720 is substantially disposed directly over the configuration 710 of magnets 712. As the magnetic ring 720, and hence, the window 714 are moved toward the magnets 712, the membrane received in the chamber 708 is generally held in place as the container 702 rotates about its axis of rotation. The extension 718 may also apply downward pressure on the membrane (e.g., via a central mask, etc.), helping preserve the flatness of the membrane received in the chamber 708.

FIG. 13C depicts a membrane holder assembly for use in an optical detection system assembly disposed within the container (also called a cartridge holder) 702. Other membrane assemblies, including those described herein, may also be received in the container/holder 702. The window 714 is disposed over the membrane assembly 200. The magnets 712 are disposed in recesses 770 in a bottom surface 772 of the container 702. As described above, when the membrane assembly 200 is received in the chamber 708, the magnets 712 pull ring 720 of the window 714 toward the surface 772 of the container/cartridge holder 702, thereby holding the membrane assembly 200 in place, as depicted in FIG. 13D.

The container/holder 702 can then be placed on a disk or chuck 780 that has a shaft 782 and a driver mechanism 784 that engages a recess defined by the base of the container/holder 702. The shaft 782 engages with the notch 704. The disk/chuck 780 fits on a motor shaft of the detection system. Rotation of the motor shaft drives the rotation of the membrane assembly 200. The shaft 782 and the driver 784 prevent the container/holder 702 from slipping or sliding on the surface of the disk 780. In addition, the magnets 786 align the container/cartridge holder 702 with a predetermined position on the surface of the disk 780, thereby facilitating registration of the initial orientation of the membrane assembly 200. Such registration can be beneficial when mapping the location of any fluorescence events (e.g., light emitted by viable cells, non-viable cells or particles).

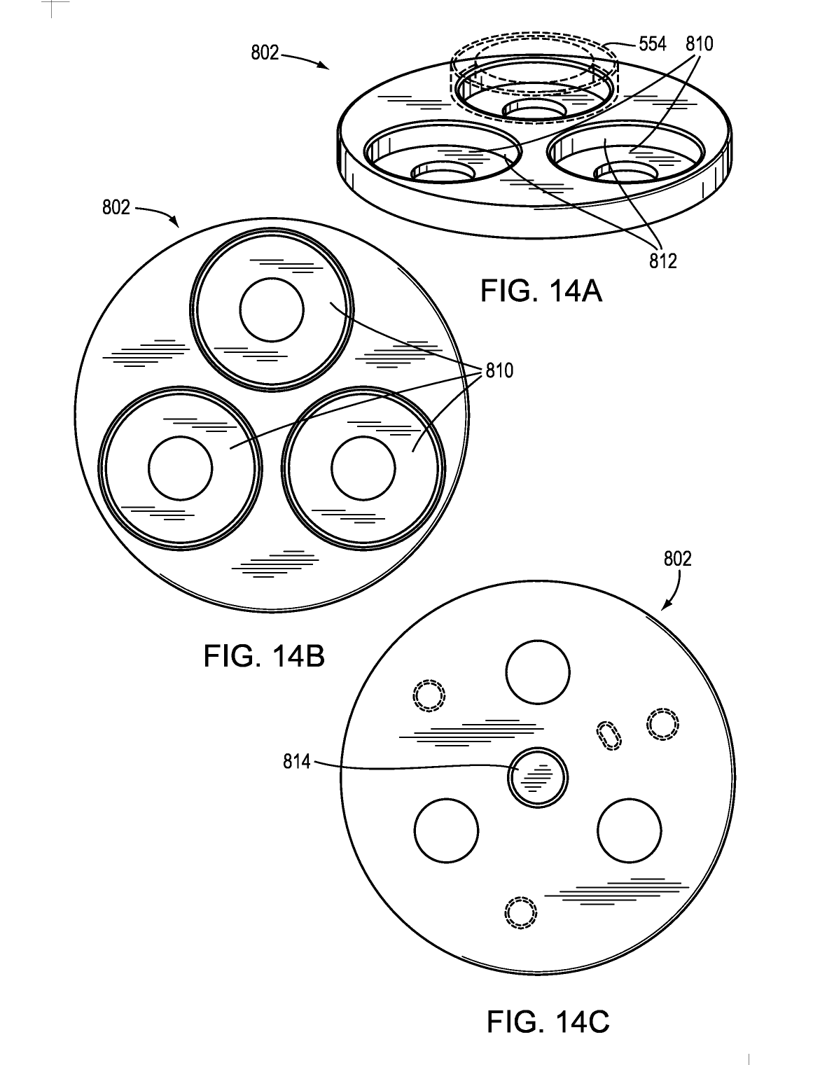

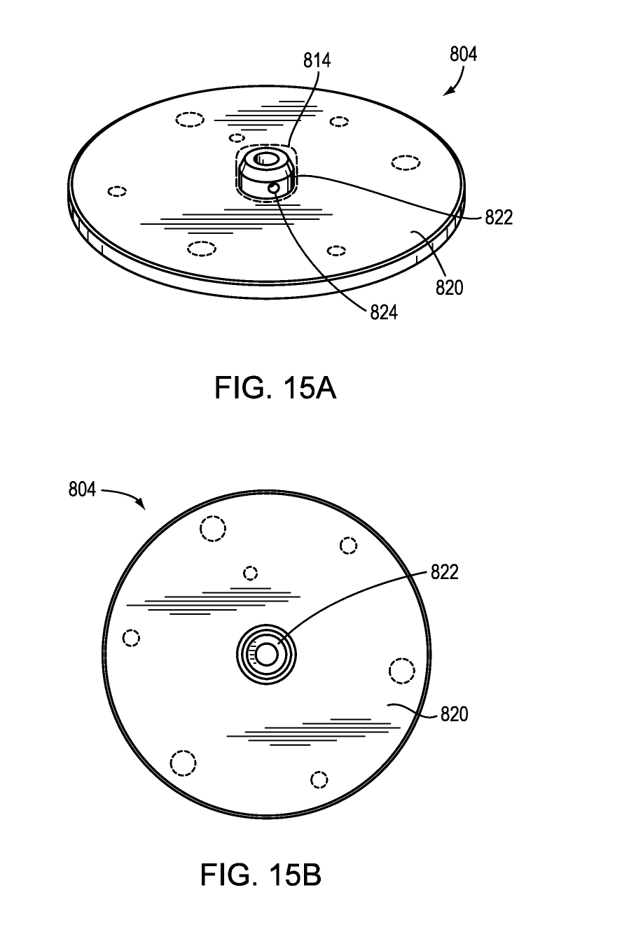

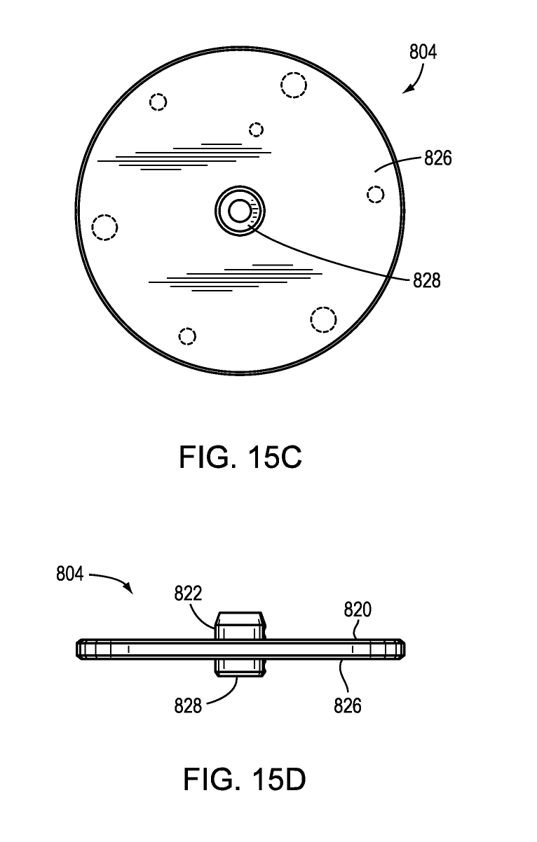

In order to minimize the number of manipulation steps for transferring the porous membrane assembly 200 into the membrane holder, which can increase the risk of contaminating the membrane assembly 200, it is contemplated that the membrane holder can be adapted to engage the membrane assembly together with the base of a cup (e.g., the base 554). FIGS. 14A-14C depict the stage 802 adapted to receive the base 554. The stage 802 may have multiple recesses 810, each adapted to receive a separate base 554. Walls 812 of the recesses are tapered to receive the similar tapered lower portion 576 of the base 554, helping ensure a secure fit for stability during rotation. The stage 802 is depicted in a substantially circular form, but may be any shape. The stage 802 may be sized to fit within the enclosure 110, either permanently or temporarily. A lower surface of the stage 810 includes a mating recess 814 for attachment to the chuck 804 (depicted in FIGS. 15A-15D). The chuck 804 provides a base on which the stage 802 sits, and provides the means for rotating the stage 802. The chuck 804 can be permanently installed in the enclosure 110, or may be removable. In an embodiment where the stage 802 and the chuck 804 are already disposed within the enclosure 110, only the base 554 with the saturated membrane 202 would need to be transferred into the enclosure 110 to begin operation, thus minimizing the number of handling steps. The chuck 804 has an upper surface 820 that can be sized to support a large portion of the stage 802 for increased stability during operation. A protrusion 822 on the top surface 820 is adapted to mate with the mating recess 814 of the stage 802, which is depicted in broken outline in FIG. 15A. The protrusion 822 may have an aperture 824 for receiving a fastener (e.g., a set screw) for further securing the stage 802 to the chuck 804. A bottom surface 826 of the chuck 804 has a protrusion 828 for mating with a drive for rotating the chuck 804.

As discussed above, for accurate detection and/or estimation of cells and/or particles, the porous membrane 202 should be flat, substantially horizontal, and at or about a predetermined distance from the source of the light impinged thereupon. Optionally, the membrane 202 is located at or near the focal length of the detection system 170. The thickness of the base 554 and flatness of the surface of the base 554 can affect the height and plane of the membrane 202.

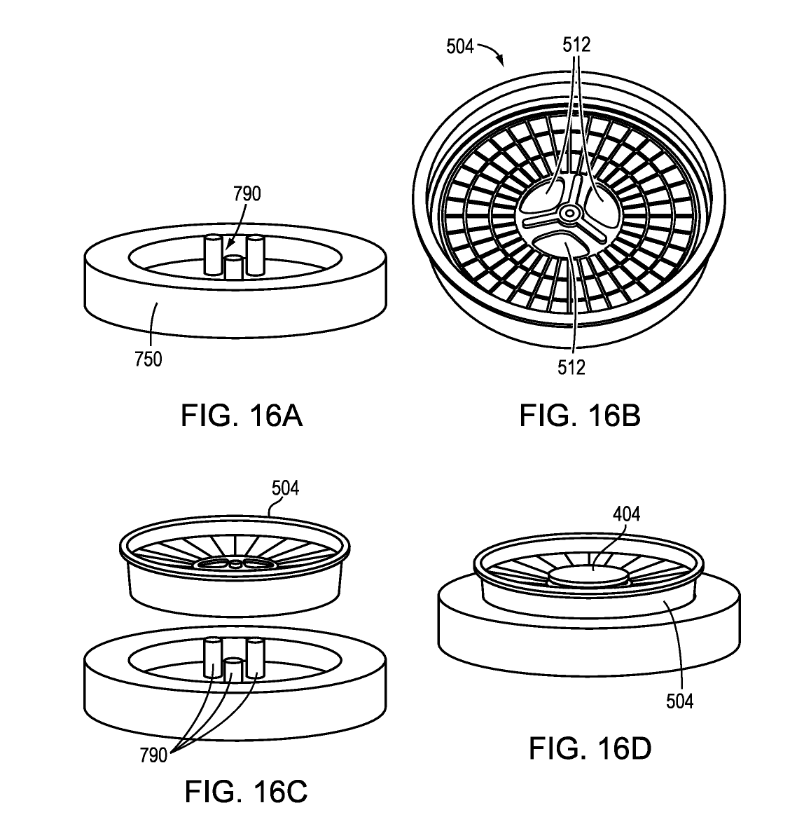

The distance and planarity may be maintained using a variety of different approaches. In one embodiment, as depicted in FIGS. 16A-16D, when using an assembly for use in the system of FIG. 1, depicted posts 790 pass through the openings 512 in the base 504, and can contact the bottom surface of the porous member 404 disposed within the base 504. The porous member 404 is lifted from base 504 as shown in FIG. 16D. Both the top and bottom surfaces of the porous member 404 can be very flat and parallel, and disposed within the focal plane of the detection system 170. The heights of the posts 790 are precisely machined to define a horizontal plane at a predetermined height in the detection system 170, such that the posts 790 directly support the membrane support member 404, free from the base 504. Accordingly, by controlling precisely solely the thickness and flatness of the support members 404, the exposed surfaces of the membranes 202 can be reliably and repeatably positioned almost exactly at the focal plane of the detection system 170. Variability in the dimensions of the bases 504 thereby do not affect the accuracy of the detection system 170. In other words, the membrane 202 disposed upon the top surface of the porous member 404 can be located substantially at the focal plane of the detection system 170 on a consistent basis. While described with respect to the base 504, the base 554 has similar openings 586 that may be used in conjunction with the posts 790.

The systems and methods described herein can be used to detect the presence and/or quantity of viable cells (for example, prokaryotic cells or eukaryotic cells) in a liquid sample. The method can be used in combination with a cell capture system and/or an optical detection system for detecting the presence of viable cells in a cell sample. The method can be used in a method to measure the bioburden (e.g., the number and/or percentage and/or fraction of viable cells (for example, viable microorganisms, for example, bacteria, yeast, and fungi)) of a particular sample of interest.

The scanning step can comprise tracing at least one of a nested circular pattern and a spiral pattern on the porous membrane with the beam of light. It is understood that during the scanning step, the porous membrane may move (for example, via linear translation) while the detection system remains static. Alternatively, the detection system may move (for example, via linear translation) while the porous membrane rotates about a single point (i.e., the porous membrane rotates about a single rotational axis). Alternatively, it is possible that both the porous membrane and the detection may move and that their relative positions are measured with respect to one another.

During operation, the membrane holder 700 and the membrane are rotated at a constant speed, and the speed can range from about 1 rpm to about 5,000 rpm, from about 1 rpm to about 1,000 rpm, from about 1 rpm to about 750 rpm, from about 1 rpm to about 500 rpm, from about 1 rpm to about 400 rpm, from about 1 rpm to about 300 rpm, from about 1 rpm to about 200 rpm, from about 1 rpm to about 100 rpm, from about 1 rpm to about 50 rpm, 20 rpm to about 5,000 rpm, from about 20 rpm to about 1,000 rpm, from about 20 rpm to about 750 rpm, from about 20 rpm to about 500 rpm, from about 20 rpm to about 400 rpm, from about 20 rpm to about 300 rpm, from about 20 rpm to about 200 rpm, from about 20 rpm to about 100 rpm, from about 20 rpm to about 50 rpm, 30 rpm to about 5,000 rpm, from about 30 rpm to about 1,000 rpm, from about 30 rpm to about 750 rpm, from about 30 rpm to about 500 rpm, from about 30 rpm to about 400 rpm, from about 30 rpm to about 300 rpm, from about 30 rpm to about 200 rpm, from about 30 rpm to about 100 rpm, or from about 30 rpm to about 50 rpm. In certain embodiments, the membrane is rotated at 200-400 rpm, for example, 300 rpm.

Similarly, the rotating membrane may be translated relative to the detection system at a constant linear velocity, which may or may not be dependent on the rotational speed. The linear velocity can vary from about 0.01 mm/min to about 20 mm/min, from about 0.01 mm/min to about 10 mm/min, from about 0.01 mm/min to about 5 mm/min, from about 0.01 mm/min to about 2 mm/min, from about 0.01 mm/min to about 1 mm/min, from about 0.01 mm/min to about 0.5 mm/min, from about 0.06 mm/min to about 20 mm/min, from about 0.06 mm/min to about 10 mm/min, from about 0.06 mm/min to about 5 mm/min, from about 0.06 mm/min to about 2 mm/min, from about 0.06 mm/min to about 1 mm/min, from about 0.06 mm/min to about 0.5 mm/min, from about 0.1 mm/min to about 20 mm/min, from about 0.1 mm/min to about 10 mm/min, from about 0.1 mm/min to about 5 mm/min, from about 0.1 mm/min to about 2 mm/min, from about 0.1 mm/min to about 1 mm/min, from about 0.1 mm/min to about 0.5 mm/min, from about 0.6 mm/min to about 20 mm/min, from about 0.6 mm/min to about 10 mm/min, from about 0.6 mm/min to about 5 mm/min, from about 0.6 mm/min to about 2 mm/min, or from about 0.6 mm/min to about 1 mm/min.

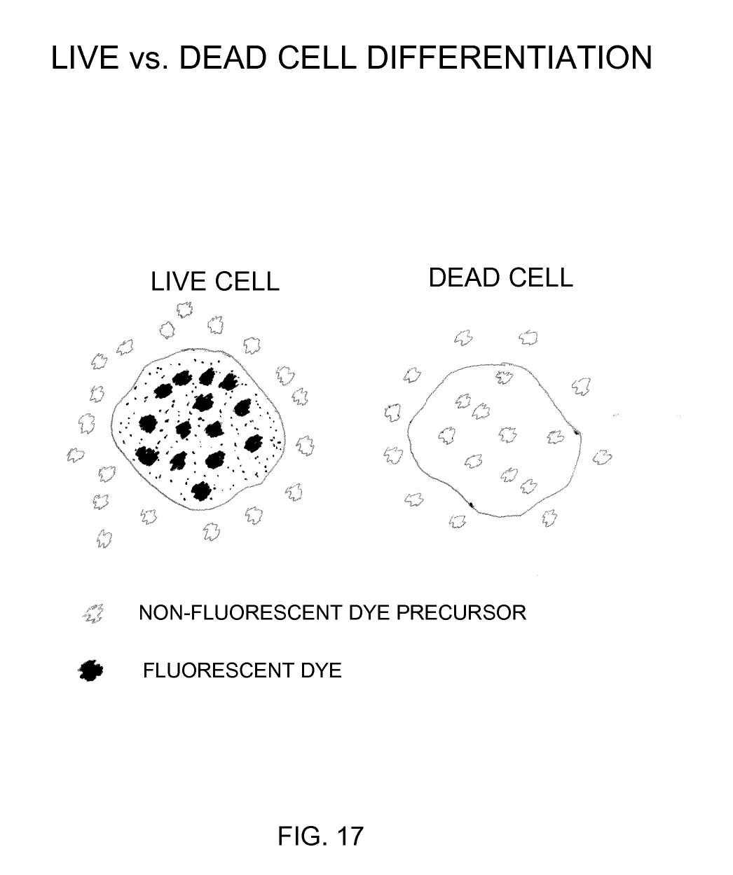

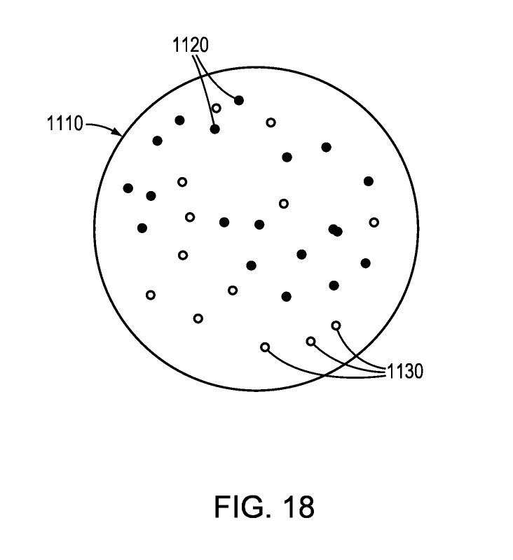

An illustrative view of cells stained by the methods described herein (e.g. as presented schematically in FIG. 17) is shown in FIG. 18. A region 1110 being interrogated by the detection system contains bright viable cells 1120 and dark non-viable cells 1130. The number, magnitude and location of the fluorescent events can be captured digitally and represented in a form that permits the operator to quantify (for example to determine the number of, percentage of) viable cells in a sample and/or otherwise to determine the bioburden of a particular sample.

As noted above, in certain embodiments, the cell capture system, the staining method, and the detection step can include or use a plurality of detectable particles, for example, fluorescent particles. The particles can be used as part of a positive control system to ensure that one or more of the cell capture system, the cell capture method, the detection system, and the method of detecting the viable cells are operating correctly. The fluorescent particles can be adapted to be excited by light having a wavelength at least in a range from about 350 nm to about 1000 nm. For example, the wavelength is at least in one range from about 350 nm to about 600 nm, from about 400 nm to about 650 nm, from about 450 nm to about 700 nm, from about 500 nm to about 750 nm, from about 550 nm to about 800 nm, from about 600 nm to about 850 nm, from about 650 nm to about 900 nm, from about 700 nm to about 950 nm, from about 750 to about 1000 nm. Certain ranges include from about 350 nm to about 600 nm and from out 600 nm to about 750 nm.

Depending upon the design of the cell capture system, the particles can be pre-disposed upon at least a portion of the porous membrane or disposed within a well formed in a mask associated with the membrane. Alternatively, the particles (for example, the fluorescent particles) can be mixed with a liquid sample prior to passing the sample through the porous membrane. In such an approach, the fluorescent particles can be dried in a vessel that the sample of interest is added to. Thereafter, the particles can be resuspended and/or dispersed within the liquid sample. Alternatively, the fluorescent particles can be present in a second solution that is mixed with the sample of interest. Thereafter, the particles can dispersed within the liquid sample. The particles, for example, a plurality of particles, can then be captured on the porous membrane along with the cells in the cell sample, which acts as a positive control for the cell capture system. The particles, for example, the fluorescent particles, can be detected once they emit a fluorescent event upon activation by light from the light source.

Using the staining protocols described herein, it is possible to determine the number of viable cells in at least a portion of the cell sample, for example, a liquid sample. The liquid sample can be, for example, a water sample, a comestible fluid (e.g., wine, beer, milk, baby formula or the like), a body fluid (e.g., blood, lymph, urine, cerebrospinal fluid or the like), growth media, a liquid sample produced by harvesting cells from a source of interest (e.g., via a swab) and then dispersing and/or suspending the harvested cells, if any, a liquid sample, for example, buffer or growth media. Furthermore, the detection system can be used to determine the location(s) of the viable cells on the permeable membrane, as described above.

After the detection step, the viable cells can be cultured under conditions that permit growth and/or proliferation of the viable cells (e.g., microorganisms) captured by the porous membrane. The genus and/or species of the viable organisms can be determined by standard procedures, for example, microbiological staining and visualization procedures, or molecular biological procedures, for example, amplification procedures including polymerase chain reaction, ligase chain reaction, rolling circle replication procedures, and the like, and by nucleic acid sequencing.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

### EXAMPLES

The invention now being generally described, will be more readily understood by reference to the following examples, which are included merely for purposes of illustration of certain aspects and embodiments of the present invention, and are not intended to limit the scope of the invention in any way.

### Example 1

### Imaging of Viable Microbes (E. coli) on a Rotating Membrane Using Fluorescein Diacetate 5-Maleimide, an Esterase Substrate

This Example demonstrates that it is possible to selectively stain and image viable microbes with fluorescein diacetate 5-maleimide on a solid membrane support using a detection system shown schematically in FIG. 1A.

Viable E. coli cells were prepared by picking a colony cultured on a conventional media plate and then transferring the cells into Phosphate Buffered Saline (PBS). The cells then were suspended by vortexing and then were further diluted in PBS to give a turbidity equivalent to a 1.0 McFarland standard. One microliter of this suspension was then diluted into 100 milliliters of PBS and filtered through a 0.47 μm gold sputtered PET membrane (Sabeu-Northeim, Germany) by using a vacuum system to capture the cells on the membrane. The cells were captured upon a porous membrane disposed upon a porous support member, for example, as shown in FIGS. 4A and 4B, by passing the solution through the membrane and porous support member.

Fluorescein diacetate 5-maleimide (Sigma-Aldrich, St. Louis, Mo.) was prepared as a 5 mM stock solution in dimethylsulfoxide (Sigma-Aldrich, St. Louis, Mo.) containing 10% w/v poloxamer 407 (Sigma-Aldrich, St. Louis, Mo.). A working stain solution was prepared by diluting one microliter of the stock solution into one milliliter of 2-(N-morpholino) ethanesulfonic acid buffered saline (MES) pH 4.7 (Thermo Scientific—Rockford, Ill.). The working stain solution was then applied to the cells captured upon the porous membrane and incubated for 30 minutes at 37° C.

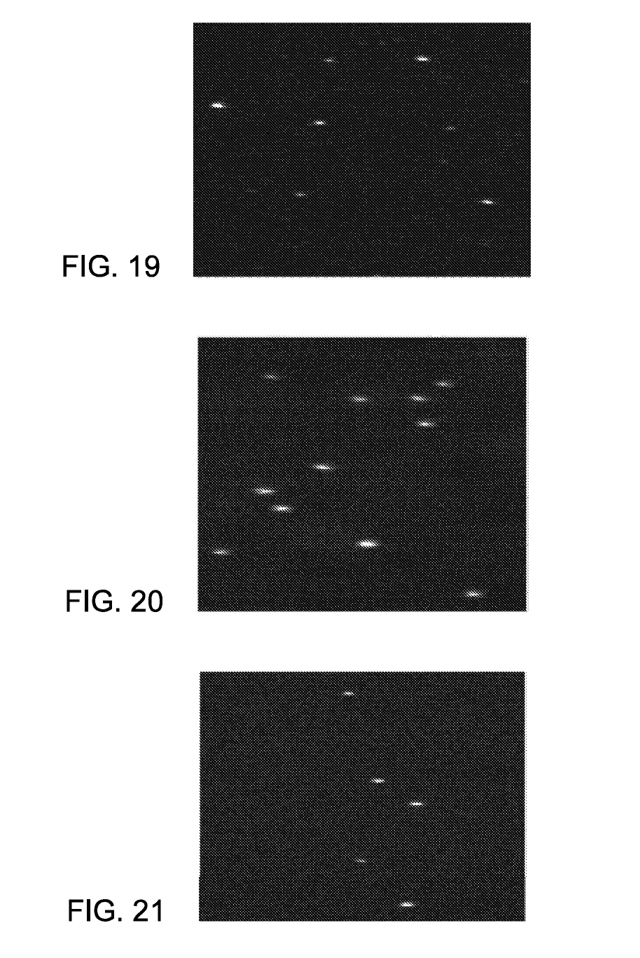

The resulting membrane then was transferred to the platform of a detection system shown schematically in FIG. 1. The membrane was rotated at 5 revolutions per second (300 revolutions per minute), and the fluorescent events were detected via the detection system. FIG. 19 shows a portion of the scanned membrane surface in which the viable E. coli cells are clearly visible as bright fluorescent events. FIG. 19 represents the captured fluorescence emission from 490 nm-540 nm. To confirm that the bright fluorescent events were due to viable cells, a control experiment was performed in which the viable cell solution was replaced by sterile PBS and then stained as stated above. When scanned, the control experiment resulted in a dark field without any bright fluorescent events.

### Example 2

### Imaging of Viable Microbes (Micrococcus luteus) on a Rotating Membrane Using 5(6)-Carboxyfluorescein Diacetate N-Succinimidyl Ester, an Esterase Substrate

This Example demonstrates that it is possible to selectively stain and image viable microbes with 5(6)-Carboxyfluorescein diacetate N-succinimidyl ester on a solid membrane support using a detection system shown schematically in FIG. 1A.

Viable Micrococcus luteus cells were prepared by picking a colony cultured on a conventional media plate and then transferring the cells into Phosphate Buffered Saline (PBS). The cells then were suspended by vortexing and then were further diluted in PBS to give a turbidity equivalent to a 1.0 McFarland standard. One microliter of this suspension was then diluted into 100 milliliters of PBS and filtered through a 0.47 μm gold sputtered PET membrane (Sabeu—Northeim, Germany) by using a vacuum system to capture the cells on the membrane. The cells were captured upon a porous membrane disposed upon a porous support member, for example, as shown in FIGS. 4A and 4B, by passing the solution through the membrane and porous support member.

5(6)-Carboxyfluorescein diacetate N-succinimidyl ester (Sigma-Aldrich, St. Louis, Mo.) was prepared as a 5 mM stock solution in dimethylsulfoxide (Sigma-Aldrich, St. Louis, Mo.) containing 10% w/v poloxamer 407 (Sigma-Aldrich, St. Louis, Mo.). A working stain solution was prepared by diluting one microliter of stock solution into one milliliter of 2-(N-morpholino) ethanesulfonic acid buffered saline (MES) pH 4.7 (Thermo Scientific—Rockford, Ill.). The working stain solution was applied to the cells captured upon the porous membrane and incubated for 30 minutes at 37° C.

The resulting membrane then was transferred to the platform of a detection system shown schematically in FIG. 1. The membrane was rotated at 5 revolutions per second (300 revolutions per minute), and the fluorescent events were detected via the detection system. FIG. 20 shows a portion of the scanned surface in which the viable population of Micrococcus luteus cells are clearly visible as bright fluorescent events. FIG. 20 represents the captured fluorescence emission from 490 nm-540 nm. To confirm these bright fluorescent events were due to viable cell staining, a control experiment was performed in which the viable cell solution was replaced by sterile PBS and then stained as stated above. When scanned, the control experiment resulted in a dark field without any bright fluorescent events.

### Example 3

### Imaging of Viable Microbes (Staphylococcus aureus) on a Rotating Membrane Using 5-Cyano-2,3-Ditolyl Tetrazolium Chloride, a Bioactivatable Tetrazolium Dye

This Example demonstrates that it is possible to selectively stain and image viable microbes with 5-Cyano-2,3-ditolyl tetrazolium chloride (CTC) on a solid membrane support using a detection system shown schematically in FIG. 1A.

Viable Staphylococcus aureus cells were prepared by picking a colony cultured on a conventional media plate and then transferring the cells into Phosphate Buffered Saline (PBS). The cells then were suspended by vortexing and then were further diluted in PBS to give a turbidity equivalent to a 1.0 McFarland standard. One microliter of this suspension was then diluted into 100 mL of PBS and filtered through a 0.4 μm gold sputtered PET membrane (Sabeu—Northeim, Germany) by using a vacuum system to capture the cells on the membrane. The cells were captured upon a porous membrane disposed upon a porous support member, for example, as shown in FIGS. 4A and 4B, by passing the solution through the membrane and porous support member.

CTC (Sigma-Aldrich, St. Louis, Mo.) was prepared as a 5 mM solution in Hank's Buffered Salt Solution (HBSS) (Sigma-Aldrich, St. Louis, Mo.) containing 1 mM potassium ferricyanide (Sigma-Aldrich, St. Louis, Mo.). The stain solution was applied to the cells captured upon the porous membrane and incubated for 30 minutes at 37° C.

The resulting membrane was then transferred to the platform of a detection system shown schematically in FIG. 1. The membrane was rotated at 5 revolutions per second (300 revolutions per minute), and the fluorescent events were detected via the detection system. FIG. 21 shows a small portion of the scanned surface in which the viable population of Staphylococcus aureus cells are clearly visible as bright fluorescent events. FIG. 21 represents the captured fluorescence emission from 590 nm-700 nm. To verify these bright fluorescent events were due to viable cells, a control experiment was performed in which the viable cell solution was replaced by sterile PBS and then stained as stated above. When scanned, the control experiment resulted in a dark field without any bright fluorescent events.

### Example 4

### Imaging of Viable E. coli Cells or Microcolonies of E. coli on a Rotating Membrane Using 5-Cyano-2,3-Di-(p-Tolyl)Tetrazolium Chloride, a Bioactivatable Tetrazolium Dye

This Example demonstrates that it is possible to selectively stain and image viable microcolonies with 5-Cyano-2,3-di-(p-tolyl)tetrazolium chloride (CTC) on a solid membrane support using a detection system shown schematically in FIG. 1A. This Example also demonstrates the increase in fluorescent intensity that can be achieved by staining microcolonies of cells rather than individual cells.

Viable E. coli cells were prepared by picking a colony cultured on a conventional media plate and then transferring the cells into PBS. The cells then were suspended by vortexing and then were further diluted in PBS to give a turbidity equivalent to a 1.0 McFarland standard. One microliter of this suspension was then diluted into 10 mL of PBS. One hundred microliters of this solution was further diluted into 100 mL of PBS and filtered through a 0.4 μm black PET membrane (Sabeu—Northeim, Germany) by using a vacuum system to capture the cells on the membrane. The cells were captured upon a porous membrane disposed upon a porous support member, for example, as shown in FIGS. 4A and 4B, by passing the solution through the membrane and porous support member.

The resulting membrane was then placed upon a conventional tryptic soy agar plate and incubated for 6 hours at 32° C. The membrane was then removed from the agar plate and CTC dye (Sigma-Aldrich, St. Louis, Mo.) was applied to the surface of the agar plate as follows. CTC stain was prepared as a 9.6 mM solution in 0.9% saline solution (Sigma-Aldrich, St. Louis, Mo.) containing 40 μM Menadione (Sigma-Aldrich, St. Louis, Mo.). Then 50 μL of the stain solution was applied onto the agar plate used previously for incubation. The membrane on which the cells were captured was then replaced upon the droplet of CTC stain and the membrane incubated for additional 30 minutes at 32° C.

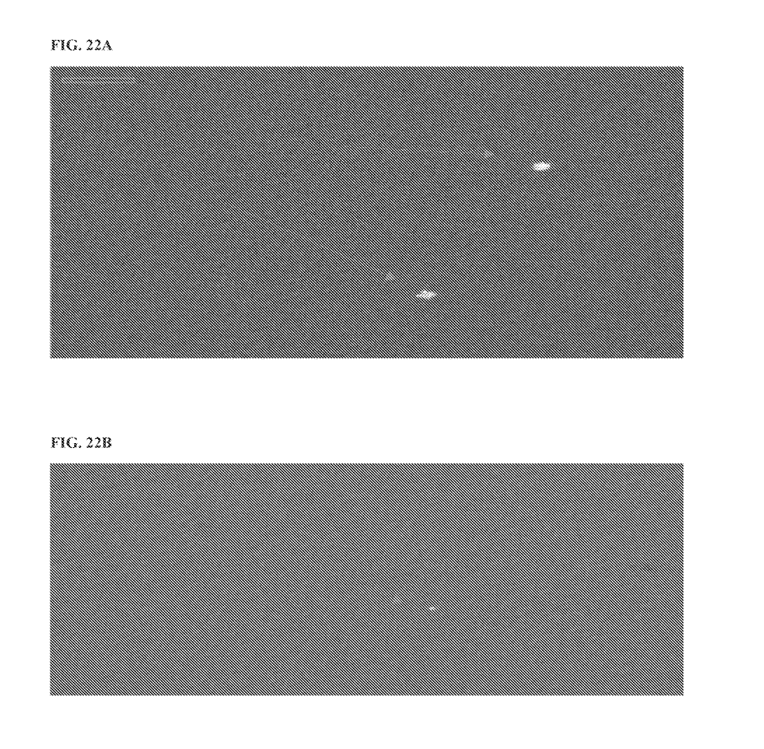

The resulting membrane then was transferred to the platform of a detection system shown schematically in FIG. 1. The membrane was rotated at 5 revolutions per second (300 revolutions per minute), and the fluorescent events were detected via the detection system. FIG. 22A shows a portion of the scanned membrane surface in which the viable E. coli microcolonies are clearly visible as bright fluorescent events. FIG. 22A represents the captured fluorescence emission from 610 nm-640 nm. To confirm that the bright fluorescent events were due to viable microcolonies, a control experiment was performed in which the viable cell solution was replaced by sterile PBS and then incubated and stained as stated above. When scanned, the control experiment resulted in a dark field without any bright fluorescent events.

The benefit of culturing the cells to form microcolonies prior to staining is shown by comparing the size and intensity of the fluorescent events produced by microcolonies (FIG. 22A) versus single cells (FIG. 22B). FIG. 22B shows a fluorescence image of an individual cell stained with CTC and detected as described above. The results show that incubating the captured cells for 6 hours at 32° C. with media allow the cells to multiply several times, increasing the number of cells present to form a microcolony. An unintended benefit was noticed when comparing a microcolony to an individual cell stained directly. In FIG. 22B, a single cell is highlighted by the arrow. This is clearly smaller and less intense than the microcolonies seen in FIG. 22A. In addition to this, it was noticed that non-cellular debris can be the same size and intensity as individual cells and may be counted as “false positives”. By growing the single cells to microcolonies, non-cellular debris is more easily distinguished from “true positives,” giving a more reliable and accurate cell count of viable cells.

### INCORPORATION BY REFERENCE

The entire disclosure of each of the patent documents and scientific articles referred to herein is incorporated by reference for all purposes. The entire description of U.S. Provisional Patent Application Ser. No. 61/641,805; 61/641,809; 61/641,812; 61/784,759; 61/784,789; and 61/784,807, U.S. Patent Publication Nos US2013/0316394, US2013/0309686, and US2013/0323745, and International Patent Application Nos. PCT/US2013/039347, PCT/US2013/039349 and PCT/US2013/39350, are each are incorporated by reference herein for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Various structural elements of the different embodiments and various disclosed method steps may be utilized in various combinations and permutations, and all such variants are to be considered forms of the invention. Scope of the invention is thus indicated by the appended claims rather than by the foregoing description, and all changes that come within the meaning and range of equivalency of the claims are intended to be embraced therein.

What is claimed is:

1. A method of detecting the presence and/or quantity of viable cells in a liquid sample, the method comprising the steps of:
(a) exposing viable cells, if any, retained by at least a portion of a substantially planar porous membrane after passing the liquid sample therethrough with a dye precursor selected from the group consisting of a bioactivatable tetrazolium dye and an esterase-activatable dye, under conditions so that the dye precursor is converted to a fluorescent label by a viable cell;
(b) scanning the portion of the porous membrane by rotating the porous membrane relative to a detection system comprising,
(i) a light source emitting a beam of light of a wavelength adapted to excite the fluorescent label to produce an emission event, and
(ii) at least one detector capable of detecting the emission event, thereby to interrogate a plurality of regions of the planar porous membrane and to detect emission events produced by excitation of fluorescent label associated with any viable cells; and
(c) determining the presence and/or quantity of viable cells captured by the membrane based upon the emission events detected in step (b).

2. The method of claim 1, wherein, in step (a), the cells are labeled using a bioactivatable tetrazolium dye.

3. The method of claim 1 or 2, wherein the beam of light source emits light having a wavelength in a range of from about to 350 nm to about 1000 nm.

4. The method of claim 3, wherein the wavelength is at least in one range from about 350 nm to about 600 nm and from out 600 nm to about 750 nm.

5. The method of any one of claims 1-4, wherein the detector detects emitted light in a range of from about to 350 nm to about 1000 nm.

6. The method of claim 5, wherein the optical detector detects emitted light in at least one range selected from about 350 nm to about 450 nm, from about 450 nm to about 550 nm, from about 550 nm to about 650 nm, from about 650 nm to about 750 nm, from about 750 nm to about 850 nm, and from about 850 nm to about 950 nm.

7. The method of any one of claims 1-6, wherein the porous membrane comprises a disc.

8. The method of any one of claims 1-7, wherein the porous membrane is substantially non-autofluorescent when exposed to light having a wavelength in the range from about 350 nm to about 1000 nm.

9. The method of any one of claims 1-8, wherein the porous membrane has a flatness tolerance of up to about 100 μm.

10. The method of any one of claims 1-9, wherein the porous membrane defines a plurality of pores having an average diameter less than about 1 μm so as to permit fluid to traverse the porous membrane while retaining cells thereon.

11. The method of any one of claims 1-10, wherein the porous membrane has a thickness in a range selected from the group consisting of from 1 μm to 3,000 μm; from 10 μm to 2,000 μm; and from 100 μm to 1,000 μm.

12. The method of any one of claims 1-11, wherein the porous membrane is disposed upon a fluid permeable support member.

13. The method of claim 12, wherein the support member has a thickness in a range selected from the group consisting of from 0.1 mm to 10 mm; from 0.5 mm to 5 mm; and from 1 mm to 3 mm.

14. The method of any of claims 1-13 further comprising capturing on the porous membrane a plurality of fluorescent particles that emit a fluorescent event upon activation by light from the light source.

15. The method of any one of claims 1-14 further comprising determining the quantity of viable cells in at least a portion of the liquid sample.

16. The method of any one of claims 1-15 further comprising determining locations of the viable cells on the permeable membrane.

17. The method of any one of claims 1-16 further comprising, prior to step (a), culturing the porous membrane under conditions that permit growth and/or proliferation of the viable cells captured on the membrane to form cell colonies.

18. The method of any one of claims 1-17 further comprising, during step (a), culturing the porous membrane disposed upon growth media containing the dye precursor and/or having the dye precursor disposed upon a surface adjacent the porous membrane for a time to permit the dye precursor to permeate the membrane and enter viable cells disposed upon the membrane.

19. The method of any one of claims 1-18 further comprising, after step (c), culturing the porous membrane under conditions that permit growth and/or proliferation of the viable cells captured by the porous membrane.

20. The method of any one of claims 1-19, wherein the viable cells are microorganisms.

21. The method of any one of claims 1-20 further comprising identifying a genus and/or species of the viable cells.

22. The method of any one of claims 1-21, wherein the scanning step (b) comprises tracing at least one of a nested circular pattern and a spiral pattern on the porous membrane with the beam of light.

23. The method of any one of claims 1-22, wherein the viable cells are cultured under conditions to permit cell proliferation prior to step (a), during step (a), or prior to and during step (a).

24. The method of claim 23, wherein the viable cells disposed upon the porous membrane are cultured under conditions to permit cell proliferation.

25. The method of any one of claims 1-22, wherein the viable cells are cultured under conditions to permit cell proliferation after step (a) but prior to step (b).

26. The method of any one of claims 1-25, wherein the bioactivatable tetrazolium dye is represented by Formula I: 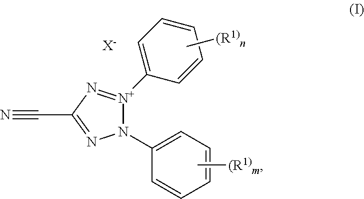
wherein:
X is halogen or ⁻ OC(O)R³;
R¹ and R² each represent independently for each occurrence hydrogen, C₁-C₆ alkyl, C₃-C₆ cycloalkyl, C₁-C₆ alkylene-(C₃-C₆ cycloalkyl), halogen, C₁-C₆ haloalkyl, hydroxyl, C₁-C₆ alkoxyl, —O—(C₃-C₆ cycloalkyl), nitro, cyano, —C(O)R³, —CO₂R³, —C(O)N(R⁴)₂, or —N(R⁴)C(O)R³;
R³ represents independently for each occurrence C₁-C₆ alkyl, C₁-C₆ haloalkyl, C₃-C₆ cycloalkyl, C₁-C₆ alkylene-(C₃-C₆ cycloalkyl), aryl, or heteroaryl;
R⁴ represents independently for each occurrence hydrogen, C₁-C₆ alkyl, C₃-C₆ cycloalkyl, C₁-C₆ alkylene-(C₃-C₆ cycloalkyl), or two occurrences of R⁴ attached to the same nitrogen atom are taken together with the nitrogen atom to which they are attached to form a 3-7 membered heterocyclic ring; and
m and n each represent independently 1, 2, or 3.

27. The method of claim 26, wherein R¹ and R² each represent independently for each occurrence hydrogen, C₁-C₆ alkyl, or C₃-C₆ cycloalkyl.

28. The method of claim 26 or 27, wherein m and n are 1.

29. The method of any one of claims 1-25, wherein the bioactivatable tetrazolium dye is represented by Formula I-A: 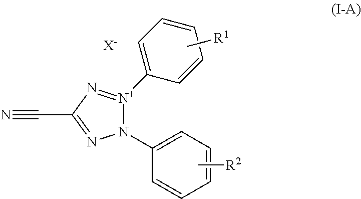
wherein: X is halogen; and R¹ and R² each represent independently C₁-C₆ alkyl, C₃-C₆ cycloalkyl, or C₁-C₆ haloalkyl.

30. The method of any one of claims 1-25, wherein the bioactivatable tetrazolium dye is 5-cyano-2,3-di-(p-tolyl)tetrazolium halide.

31. The method of any one of claims 1-25, wherein the bioactivatable tetrazolium dye is 5-cyano-2,3-di-(p-tolyl)tetrazolium chloride.

32. The method of any one of claims 1 or 3-25, wherein the esterase-activatable dye is represented by Formula II: 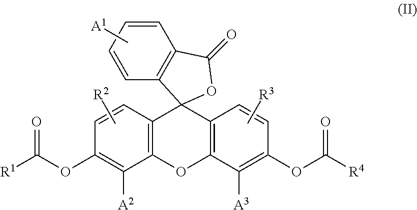
or a salt thereof, wherein:
R¹ and R⁴ each represent independently C₁-C₆ alkyl, C₃-C₆ cycloalkyl, C₁-C₆ alkylene-(C₃-C₆ cycloalkyl), C₁-C₆ haloalkyl, aryl, or heteroaryl;
R² and R³ each represent independently hydrogen, C₁-C₆ alkyl, C₃-C₆ cycloalkyl, C₁-C₆ alkylene-(C₃-C₆ cycloalkyl), halogen, C₁-C₆ haloalkyl, hydroxyl, or C₁-C₆ alkoxyl;
R⁵ represents independently for each occurrence hydrogen, C₁-C₆ alkyl, or C₃-C₆ cycloalkyl;
A¹ is hydrogen, 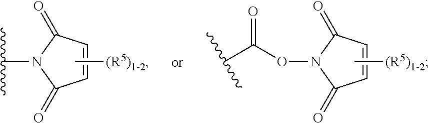
and
A² and A³ each represent independently hydrogen, C₁-C₆ alkyl, C₃-C₆ cycloalkyl, C₁-C₆ alkylene-(C₃-C₆ cycloalkyl), C₁-C₆ haloalkyl, or C₁-C₆ alkylene-N[—C₁-C₄ alkylene-CO₂—C₁-C₄ alkylene-O—C(O)—C₁-C₄ alkyl]₂.

33. The method of claim 32, wherein R¹ and R⁴ are C₁-C₆ alkyl.

34. The method of claim 32, wherein R¹ and R⁴ are methyl.

35. The method of any one of claims 32-34, wherein R² and R³ are hydrogen.

36. The method of any one of claims 32-35, wherein A¹ is 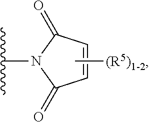
and A² and A³ are hydrogen.

37. The method of any one of claims 32-35, wherein A¹ is 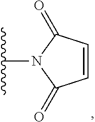 [SMILES: *N1C(=O)C=CC1=O]
and A² and A³ are hydrogen.

38. The method of any one of claims 32-35, wherein A¹ is 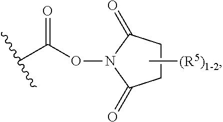
and A² and A³ are hydrogen.

39. The method of any one of claims 32-35, wherein A¹ is 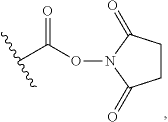 [SMILES: *C(=O)ON1C(=O)CCC1=O]
and A² and A³ are hydrogen.

40. The method of any one of claims 32-35, wherein A¹ is hydrogen, and A² and A³ are C₁-C₆ alkylene-N[—C₁-C₄ alkylene-CO₂—C₁-C₄ alkylene-O—C(O)—C₁-C₄ alkyl]₂.

41. The method of any one of claims 32-35, wherein A¹ is hydrogen, and A² and A³ are —CH₂—N[—CH₂—CO₂—CH₂—O—C(O)—CH₃]₂.

42. The method of any one of claims 1 or 3-25, wherein the esterase-activatable dye is fluorescein diacetate 5-maleimide.

43. The method of any one of claims 1 or 3-25, wherein the esterase-activatable dye is calcein-AM.

44. The method of any one of claims 1 or 3-25, wherein the esterase-activatable dye is 5-carboxyl-fluorescein diacetate N-succinimidyl ester.
